(19) United States
(12) Patent Application Publication
(10) Pub. No.: US 2025/0299819 A1
(43) Pub. Date: Sep. 25, 2025

(54) METHODS FOR PREDICTING A RESPONSE TO IMMUNOTHERAPY

(71) Applicant: The Regents of the University of California, Oakland, CA (US)

(72) Inventors: Hannah Kathryn Carter, San Diego, CA (US); Timothy Sears, La Jolla, CA (US); Maurizio Zanetti, La Jolla, CA (US)

(21) Appl. No.: 19/083,613

(22) Filed: Mar. 19, 2025

Related U.S. Application Data
(60) Provisional application No. 63/567,207, filed on Mar. 19, 2024.

(51) Int. Cl. G16H 50/20; G16B 30/00; G16B 40/20; G16H 10/40; G16H 20/10; G16H 70/40

(52) U.S. Cl. CPC G16H 50/20; G16B 30/00; G16B 40/20; G16H 10/40; G16H 20/10; G16H 70/40

(57) ABSTRACT

Disclosed herein are methods and systems for predicting a subject's response to immunotherapy to treat a cancer, including receiving sequencing data of the subject; determining, using the sequencing data, a plurality of somatic features for the subject and a plurality of germline features for the subject; generating, using the plurality of somatic features for the subject and the plurality of germline features for the subject, an immune checkpoint blockade (ICB) response score for the subject to represent a likelihood of response to an immunotherapy for the subject; and comparing the ICB response score for the subject to an ICB response threshold value.

### CLAIM OF PRIORITY

This application claims the benefit of U.S. Provisional Application Ser. No. 63/567,207, filed on Mar. 19, 2024. The entire contents of the foregoing are incorporated herein by reference.

### FEDERALLY SPONSORED RESEARCH OR DEVELOPMENT

This invention was made with Government support under Grant No. CA269919 awarded by the National Cancer Institute. The Government has certain rights in the invention.

### TECHNICAL FIELD

This disclosure generally relates to immunology.

### BACKGROUND

Immune checkpoint blockade (ICB) therapeutics have shifted the cancer treatment paradigm for patients who once faced limited therapeutic options. Development of ICB has led to remissions in some patients with advanced cancers. ICB is now a standard treatment in some tumor types. However, some patients still fail to benefit from the treatment while experiencing the side effects and costs of the therapeutics. Identifying those patients who would effectively respond to immunotherapy remains a challenge.

### SUMMARY

In general, an aspect disclosed herein is a computer implemented method for predicting a subject's response to an immunotherapy procedure to treat a cancer. The computer implemented method includes (a) receiving sequencing data of the subject; (b) determining, using the sequencing data, a plurality of somatic features for the subject and a plurality of germline features for the subject; and (c) generating, using the plurality of somatic features for the subject and the plurality of germline features for the subject, an immune checkpoint blockade (ICB) response score for the subject to represent a likelihood of response to an immunotherapy for the subject; (d) comparing the ICB response score for the subject to an ICB response threshold value.

Examples may include one or more of the following features. Determining a likelihood of response to an immunotherapy for the subject may include classifying the subject as an immunotherapy-responder based on a determination that the ICB response score is greater than the ICB response threshold. Determining a likelihood of response to an immunotherapy for the subject may include classifying the subject as an immunotherapy-nonresponder based on a determination that the ICB response score may be less than the ICB response threshold. The sequencing data may be whole-exome sequencing data. The plurality of sequencing feature values may include a plurality of somatic feature values, a plurality of germline feature values, or both. The plurality of somatic feature values may include at least one of the group of: an immunoediting feature value, an immune escape feature value, an intratumoral heterogeneity feature value, a tumor mutational burden (TMB) feature value, a measure of immune evasion feature value, a damage of MHC-I alleles feature value, a DNA based t cell infiltration feature value, a somatic mutation of genes in an antigen presentation pathway feature value, an intratumoral heterogeneity feature value, and a fraction of TMB subclonal feature value. The plurality of germline features may include at least one of the group of: a single-nucleotide polymorphisms (SNP) associated with an immune infiltration levels feature value, a DNA repair and replication feature value, an immune signaling feature value, and an antigen processing and presentation feature value. The SNP associated with the immune infiltration levels may be an SNP associated with FCGR2B, CTSS, FAM167A, FPR1, PDCD1, ITGB2, CTSW, FCGR3B, GPLD1, DCTN5, ERAP1, VAMP8, VAMP3, LYZ, ERAP2, DHFR, or TREX1 gene. The sequencing data may include RNA sequencing data and the method may include (a) determining a tumor immune microenvironment (time) infiltration value from the RNA sequencing data to represent a composition of immune infiltrates. Determining the immune checkpoint blockade (ICB) response score may include use of at least one of the group may include of at least one of the plurality of somatic features, at least one of plurality of the plurality of germline features, and the time infiltration value. The composition of immune infiltrates may include at least one of the group of: an effector CD8+ t cell infiltrate level, a joint B and CD4+ t cell level, and a target checkpoint expression. The cancer may be selected from at least one of the group may include of: a bladder cancer, a breast cancer, a cervical cancer, a colon cancer, a endometrial cancer, a esophageal cancer, a fallopian tube cancer, a gall bladder cancer, a gastrointestinal cancer, a head and neck cancer, a hematological cancer, a Hodgkin lymphoma, a laryngeal cancer, a liver cancer, a lung cancer, a lymphoma, a melanoma, a mesothelioma, a ovarian cancer, a primary peritoneal cancer, a salivary gland cancer, a sarcoma, a stomach cancer, a thyroid cancer, a pancreatic cancer, a renal cell carcinoma, a glioblastoma, and a prostate cancer. The cancer may be a renal cell carcinoma (RCC), or a non-small cell lung cancer (NSCLC). The immunotherapy may include an immune checkpoint inhibitor. The immune checkpoint inhibitor may be selected from at least one of the group of: a PD-1 inhibitor, a PD-L1 inhibitor, and a CTLA-4 inhibitor. The method may include determining the sequencing features by (a) determining a feature importance for a multiplicity of sequencing features which may include more features than the plurality of sequencing features, (b) comparing the feature importance for each of the multiplicity of sequencing features to a feature importance threshold, and (i) if the feature importance for one of the multiplicity of sequencing features meets the feature importance threshold, including the sequencing feature which meets the feature importance threshold in the plurality of sequencing features. Determining the feature importance uses a Shapley additive explanations (SHAP) feature comparison model. The method may include determining, from the sequencing data, a number of mutations presented by a major histocompatibility complex class II (MHC-II) and a number of mutations presented by a major histocompatibility complex class I (MHC-I) of the subject, comparing the number of mutations presented by a major histocompatibility complex class II (MHC-II) and the number of mutations presented by a major histocompatibility complex class I (MHC-I) to an MHC mutation threshold, and, responsive to determining that the total number of mutations presented by the major histocompatibility complex class II (MHC-II) and the major histocompatibility complex class I (MHC-I) meets the MHC mutation threshold, determining a major histocompatibility complex (MHC) ratio of the subject. Determining the major histocompatibility complex (MHC) ratio of the subject may include, determining, from the sequencing data, a major histocompatibility complex (MHC) ratio of a total number of neoantigens presented by a major histocompatibility complex class II (MHC-II) of the subject divided by the total number of neoantigens presented by a major histocompatibility complex class I (MHC-I) of the subject, and, responsive to determining that the major histocompatibility complex (MHC) ratio of the subject meets a MHC ratio threshold, determining an immune checkpoint blockade (ICB) response score of the subject

In general, an aspect disclosed herein is a computing system for determining whether a subject is at risk of having or developing a cancer. The computing system includes a communication system configured to communicate over at least one data network with another computing device. The computing system includes one or more processors. The computing system includes memory storing instructions that, when executed by the processors, cause the processors to perform operations may include: (a) receiving sequencing data of the subject; (b) determining, using the sequencing data, a plurality of somatic features for the subject and a plurality of germline features for the subject; and (c) generating, using the plurality of somatic features for the subject and the plurality of germline features for the subject, an immune checkpoint blockade (ICB) response score for the subject to represent a likelihood of response to an immunotherapy for the subject; (d) comparing the ICB response score for the subject to an ICB response threshold value.

Examples may include one or more of the following features. Determining a likelihood of response to an immunotherapy for the subject may include classifying the subject as an immunotherapy-responder based on a determination that the ICB response score may be greater than the ICB response threshold. Determining a likelihood of response to an immunotherapy for the subject may include classifying the subject as an immunotherapy-nonresponder based on a determination that the ICB response score may be less than the ICB response threshold.

In general, an aspect disclosed herein is a method for treating a subject that has been diagnosed with a cancer. The method includes (a) receiving sequencing data of the subject; (b) determining, using the sequencing data, a plurality of somatic features for the subject and a plurality of germline features for the subject; and (c) generating, using the plurality of somatic features for the subject and the plurality of germline features for the subject, an immune checkpoint blockade (ICB) response score for the subject to represent subject to represent a likelihood of response to an immunotherapy for the subject; (d) comparing the ICB response score for the subject to an ICB response threshold value, and (e) administering to the subject the immunotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates an example system for presenting calculated potential for a cancer patient to benefit from an immunotherapy treatment associated with host and tumor characteristics on a user device.

FIG. 2 illustrates a flowchart of an example process for predicting a subject's response to an immunotherapy procedure to treat a cancer for a patient.

FIG. 3 illustrates a flowchart diagram illustrating a combination of germline and somatic/clinical features into a machine learning framework.

FIG. 4A illustrates a matrix chart comparing features from germline and somatic models passing secondary RFE on the Cristescu pan-cancer cohort are shown.

FIG. 4B illustrates a PCA of test statistics for each cohort resulting from linear feature associations.

FIG. 5A illustrates boxplots of medians of testing cohorts in which IC index scores are compared via Mann-Whitney U tests.

FIG. 5B illustrates the effect size between IC index of responders vs. nonresponders of each model and cohort.

FIG. 5C illustrates the ROC curves of each model's performance across testing cohorts with AUC.

FIG. 5D illustrates Kaplan-Meier plots of composite IC index score tertiles.

FIG. 5E illustrates the hazard ratio (HR) of IC index for each model from a multivariable Cox proportional hazard analysis of PFS across aggregated testing cohorts accounting for age, sex, and cohort.

FIG. 5F illustrates the positive predictive value of each model aggregated across all cohorts.

FIG. 5G illustrates the negative predictive value of each model aggregated across all cohorts.

FIG. 5H illustrates the correlation of germline immune checkpoint (IC) and somatic IC index, with somatic and germline model density plots of IC index for responders vs. nonresponders.

FIG. 6A illustrates boxplots of median expression-based immune measures from combined test samples with RNA sequencing.

FIG. 6B illustrates hazard plots of primary TIME biomarkers stratified by IC index score.

FIG. 6C illustrates Kaplan-Meier curves of predicted nonresponders stratified by TIME biomarker score.

FIG. 6D illustrates Kaplan-Meier curves of predicted responders stratified by TIME biomarker score.

FIG. 6E illustrates the confusion matrix of IC index and TIME score.

FIG. 7A illustrates the SHAP interaction scores for the top 12 interacting feature pairs.

FIG. 7B illustrates the bar plot of percent responder by interaction category for the MHC damage and TFH SNP interaction category.

FIG. 7C illustrates the schematic of MHC-based response groupings.

FIG. 7D illustrates the proportion of patients with MHC-I damage split by MHC response categories and response status.

FIG. 7E illustrates the allelic fraction of the TFHQTL faceted by MHC response categories and split by response status.

FIG. 7F illustrates the boxplot of median TLS signature expression split by MHC response categories and response status.

FIG. 8A illustrates a CIBERSORTx CD4/CD8 T-cell infiltration estimate ratios split by MHC reliance category and response status for both discovery and validation cohorts.

FIG. 8B illustrates Kaplan-Meier curves of overall survival of responders only in MHC II-reliant patients vs. MHC I-reliant patients for discovery and validation cohorts.

FIG. 8C illustrates Kaplan-Meier curves of overall survival of responders only in MHC II-reliant patients vs. MHC I-reliant patients for discovery and validation cohorts.

FIG. 8D illustrates HRs of key checkpoint molecule expression levels colored by MHC reliance status.

FIG. 8E illustrates Kaplan-Meier curves of LAG3 above/below median expression in MHC II-reliant patients in discovery and validation cohorts.

FIG. 8F illustrates Kaplan-Meier curves of LAG3 above/below median expression in MHC II-reliant patients in discovery and validation cohorts.

FIG. 9A illustrates a method for aggregating correlated germline quantitative trait loci into aggregate germline features for use in constructing the cIC index.

FIG. 9B illustrates a table of Pearson correlation of Gene level eQTL-scores in TCGA and Discovery cohorts.

FIG. 10 illustrates a bubble plot of HR and area under the curve (AUC) multiple genes.

FIG. 11 illustrates a plot of SHAP value versus feature value for multiple genes.

### DETAILED DESCRIPTION

Identifying biomarkers for immunotherapy response have focused on measured characteristics of the tumor or the tumor immune microenvironment (TIME). Current FDA-approved biomarkers include tumor mutation burden (TMB), microsatellite instability status, and IHC staining of the tumor microenvironment to quantify PDL1 positivity. However, these predictors of response are imperfect and their application in clinical settings is not straightforward. More measures of ICB response have been proposed, including the potential immunogenicity of somatic mutations in the tumor measures of immunoediting such as the ratio of nonsynonymous:synonymous mutations of the immunopeptidome, evidence of impaired antigen presentation quantified from somatic copy number loss and mutation of MHC genes, and tumor clone phylogeny estimates as a proxy for intratumoral heterogeneity. Some researchers successfully integrated somatic features such as these to predict ICB response using machine learning models with superior accuracy, suggesting nonlinear predictive models may capture additional biological complexity.

More recent work has uncovered a role for germline genetic variation in influencing the characteristics of the TIME and ICB response. Although whole exome sequencing (WES) methods require a matched normal tissue as a background panel for somatic mutation detection, patient germline variation has largely been ignored in the development of predictive ICB modeling, even though germline variation has a considerable effect on adaptive immune traits. Common germline variants were found to predict ICB responses independent of somatic biomarkers. Therefore, it is reasoned that although individual common variants may have a reduced influence on traits, the sum of these variations could have a large impact on the TIME. In general, cancer may arise from mutagenic processes independent of host germline genetics.

Disclosed herein is a machine learning model which integrates both somatic and germline features to identify patients who may benefit from ICB therapy. A composite model using all somatic and germline features demonstrated superior performance across multiple independent test sets relative to predictors trained on one of germline or somatic features. Analysis of the composite model revealed feature interactions that contributed to model performance, the strongest of which occurred between MHC class-I (MHC-I) damage and a germline variant associated with increased infiltration of T-follicular helper cells. Further investigation of this interaction suggested an MHC-I-independent mechanism of ICB response associated with the MHC class-II (MHC-II) CD4+ T-cell axis in some patients. Grouping ICB responders by response type showed more durable ICB responses in the MHC-II-driven response axis. For the 34% of patients with RNA expression data, characteristics of the TIME were investigated such as checkpoint expression, T-cell infiltration, and tertiary lymphoid structure (TLS) signatures. Nonlinear models using somatic, and germline features together predict ICB outcomes.

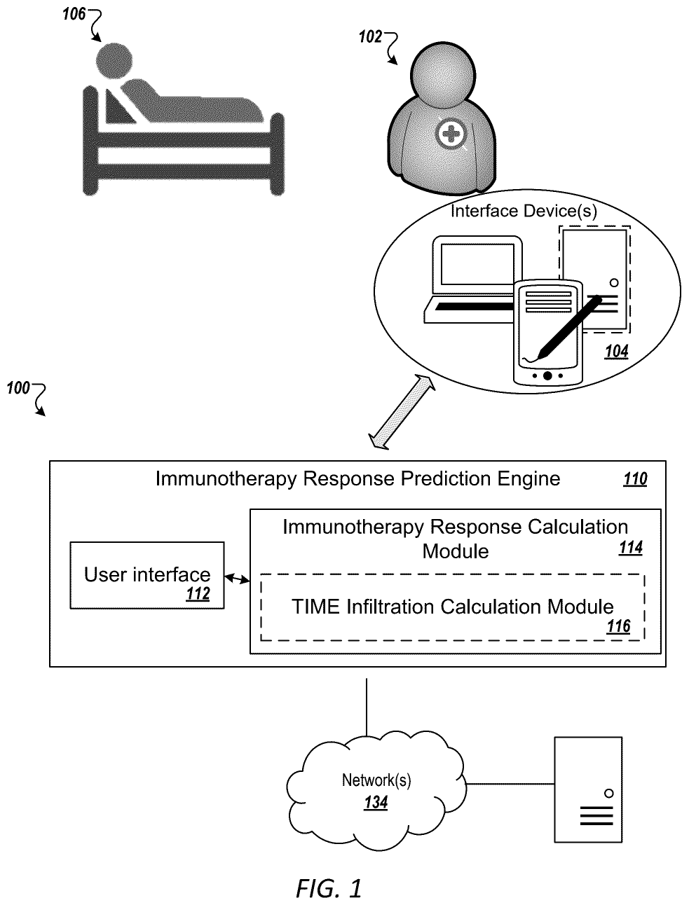

FIG. 1 is a block diagram of an example immunotherapy response prediction system 100 configured to dynamically predicting a subject's response to immunotherapy to treat a cancer based on data indicative of cellular conditions, e.g., cellular conditions data, of a patient. The example prediction system 100 can be configured to determine a likelihood of response to an immunotherapy for a patient based on a generated immune checkpoint blockade (ICB) response score.

System 100 depicts a user 102 interacting with an example interface device 104. The user 102 in this example is a clinician, e.g., a medical professional, a medical assistant, an oncologist, although the techniques disclosed in this specification may be extended for use with other users as well. In the example of FIG. 1, the user 102 is screening a patient 106 for an immunotherapy procedure to treat a cancer. The user 102 is utilizing the system 100 to determine a likelihood of response of the patient 106 to an immunotherapy, e.g., an ICB immunotherapy, based on cellular conditions of the patient 106 gathered by the user 102 pre-therapeutically.

In some examples, the cellular conditions data includes sequencing data from the patient 106. The sequencing data can include DNA sequencing data, whole genome sequencing data, whole-exome sequencing data, RNA sequencing data, or any combination of these. The sequencing data can be determined from a sample taken from the patient 106 pre-therapeutically. Cellular conditions measurement data can also include genotype arrays (SNPs).

In some implementations, cell conditions may be profiled by single cell- or epigenetic technologies. Epigenetic silencing is relevant to loss of HLA function, so these technologies may be relevant to measuring somatic features. Examples may include single cell DNA/RNA sequencing, bisulfite sequences, methylation microarrays, chromatin immunoprecipitation sequencing (ChiPseq), assay for transposase-accessible chromatin using sequencing (ATACseq), cellular indexing of transcriptomes and epitopes by sequencing (CiteSeq), or any combination of these.

Whole-exome sequencing (WES) is a genomic technique that sequences all the protein-coding regions of genes in a genome, known as exons. WES data can be used to determined genetic variants (e.g., including single nucleotide polymorphisms (SNPs) (e.g., synonymous SNPs, or nonsynonymous SNPs), insertions, deletions, and copy number variations (CNVs) within exonic regions), mutations in specific genes (e.g., missense, or nonsense mutations), mutations in tumor samples, or any combination of these.

The user 102 interacts with the interface device 104 to input data indicative of cellular conditions, e.g., cellular conditions data, into the system 100. In another example, the system 100 receives cellular conditions data from a database, look up table, or other data storage system connected to a network 134 in communication with the system 100, such as a patient data management system which stores individualized, non-modifiable risk factors specific to the patient 106. The system 100 can receive the cellular conditions data from the network 134 alone or in combination with other data input by the user 102.

The interface device 104 stores in non-transitory media the immunotherapy response calculation engine 110. The immunotherapy response calculation engine 110 includes a user interface 112 with which the user 102 interacts and inputs the cellular condition data of the patient 106 into the engine 110. The user interface 112 includes control elements for receiving the input from the user 102 such as radio buttons, text boxes, and/or other input fields into which the user 102 inputs the cellular conditions data for the patient 106.

The user interface 112 is communicatively connected to an immunotherapy response calculation module 114 which receives the cellular conditions data from the user interface 112. The module 114 can be an implementation of one or more suitable models trained to generate an output based on the received input. In some examples, the models are machine-learning models.

The immunotherapy response calculation module 114 receives as input the cellular condition data. The immunotherapy response calculation module 114 produces as output a classification of the patient 106 indicating whether the patient 106 is an immunotherapy-responder. The immunotherapy response calculation module 114 is pre-trained using a collection of cellular condition features to output classification of the patient 106 based on the cellular conditions data of the patient 106.

One example of the immunotherapy response calculation module 114 is an XGBoost model, an open-source implementation of the supervised learning, gradient-boosted trees algorithm which attempts to accurately predict a target variable by combining the estimates of a set of simpler, weaker models. The immunotherapy response calculation module 114 can also be implemented with convolutional neural networks, transformers, or other machine-learning models.

The immunotherapy response calculation module 114 is trained on patient data which included feature vectors for one or more of cellular condition data in a set of patients. The feature vectors represent a number of features of the cellular condition data determined to be predictive of a patient's response to the immunotherapy.

In some examples, the feature vectors of the cellular condition data includes somatic biomarker indicator features. A somatic biomarker indicator feature is a measurable indicator of a biological state or condition found in somatic cells (e.g., non-reproductive cells) of the patient 106. Examples of somatic biomarkers include, but are not limited to, an immunoediting biomarkers, an immune escape biomarker, an intratumoral heterogeneity biomarker, a tumor mutational burden (TMB) biomarker, a measure of immune evasion biomarker, a damage of MHC-I alleles biomarker, a DNA based T cell infiltration biomarker, a somatic mutation of genes in an antigen presentation pathway biomarker, an intratumoral heterogeneity biomarker, and a fraction of TMB subclonal biomarker.

In some examples, the feature vectors of the cellular condition data include germline biomarker indicator features. A germline biomarker indicator feature is a measurable indicator of a biological state or condition found in germline cells (e.g., reproductive cells) of the patient 106. Examples of germline biomarkers include, but are not limited to, a single-nucleotide polymorphisms (SNP) associated with an immune infiltration levels biomarker, a DNA repair and replication biomarker, an immune signaling biomarker, and an antigen processing and presentation biomarker.

In some examples, the SNP associated with the immune infiltration level biomarker is an SNP associated with an immune signaling gene, an antigen processing and presentation gene, and immune evasion gene, and immunogenicity gene, a DNA repair and replication gene, or an immune infiltration gene. Non-limiting examples include FCGR2B gene, a CTSS gene, a FAM167A gene, a FPR1 gene, a PDCD1 gene, a ITGB2 gene, a CTSW gene, a FCGR3B gene, a GPLD1 gene, a DCTN5 gene, a ERAP1 gene, a VAMP8 gene, a VAMP3 gene, a LYZ gene, a ERAP2 gene, a DHFR gene, or a TREX1 gene.

The module 114 uses the cellular conditions data to determine one or more feature values for each of the feature vectors in the model. The feature values can include one or more somatic feature values, one or more germline feature values, or both, of the patient 106. The somatic feature values can include values representing any semantic biomarker described here in. The germline feature values can include values representing any germline biomarker described herein.

The module 114 uses one or more of the somatic feature values, one or more of the germline feature values, or combinations of the somatic feature values and the germline feature values, to determine an immune checkpoint blockade response score for the patient 106. The immune checkpoint blockade response score represents a likelihood of the patient 106 to respond to an immunotherapy, e.g., an ICB therapy.

The module 114 compares the immune checkpoint blockade response score to a ICB response threshold value. The module 114 can store the ICB response threshold value in non transitory memory, or receive the ICB response threshold value from a networked location. The ICB response threshold value can be of threshold value determined previously by which the ICB response score of the patient 106 can be compared.

If the ICB response score of the patient 106 is above the ICB response threshold value, the patient may have an increased likelihood of response to an immunotherapy to treat the cancer. The module 114 outputs a classification indicating that the patient 106 is an immunotherapy-responder.

If the ICB response score of the patient 106 is below the ICB response threshold value, the patient may have a decreased likelihood of response to an immunotherapy to treat the cancer. The module 114 outputs a classification indicating that the patient 106 is an immunotherapy-non responder.

Based on the output from the engines 114, and optionally 116, the engine 110 generates an output indicative the classification of the patient 106. The engine 110 provides the output to the user interface 112 such that the immunotherapy response calculation module 114 presents the output for display to the user 102. In some examples, the engine 110 provides the output to the interface device 104. Additionally or alternatively, the engine 110 generates computer code including instructions that, when executed, cause an indication of the classification to be presented on the interface device 104.

The user 102 may administer an immunotherapy to the patient 106 in response to receiving the classification of the patient 106 as an immunotherapy responder or an immunotherapy non responder. In examples in which the patient is categorized as an immunotherapy responder, the user 102 may administer the immunotherapy to which the patient is an immunotherapy responder. In some examples the immunotherapy is an ICB immunotherapy. Administering the ICBM immunotherapy can include administering an immune checkpoint inhibitor compound to the patient 106. Non-limiting examples of the immune checkpoint inhibitor compound include a PD-1 inhibitor compound, PD-L1 inhibitor compound, and CTLA-4 inhibitor compound.

In some implementations, the cancer for which the patient 106 is being screened for immunotherapy response is a cancer which is responsive to the selected immunotherapy. In examples where the immunotherapy is an ICB immunotherapy, the cancer can be a bladder cancer, a breast cancer, a cervical cancer, a colon cancer, a endometrial cancer, a esophageal cancer, a fallopian tube cancer, a gall bladder cancer, a gastrointestinal cancer, a head and neck cancer, a hematological cancer, a Hodgkin lymphoma, a laryngeal cancer, a liver cancer, a lung cancer, a lymphoma, a melanoma, a mesothelioma, a ovarian cancer, a primary peritoneal cancer, a salivary gland cancer, a sarcoma, a stomach cancer, a thyroid cancer, a pancreatic cancer, a renal cell carcinoma, a glioblastoma, and a prostate cancer.

In some examples, the ICB response score can be scaled before comparison to the ICB response threshold value, before outputting the ICB response score to the user interface 112, or any combination of these. An example of a scaling function includes sklearn MinMaxScaler to scale the ICB response score. In some examples, the module 114 determines an ICB response score in a range from 0 to 1. The scaling function may then use the ICB response score to determine an immune checkpoint index score which is a scalar of the ICB response score. In some examples the immune checkpoint index score may be in a range from 0 to 10, non limiting. Scaling the ICB response score may aid visualization for the user 102, the patient 106, or other viewers of the module 114 output.

The immunotherapy response calculation module 114 can optionally include a tumor immune microenvironment (TIME) infiltration calculation module 116. The TIME infiltration calculation module 116 determines a TIME infiltration value based on the sequencing data, e.g., RNA sequencing data. The TIME infiltration value can also be calculated from spatial protein expression profiling (e.g., immunohistochemistry, immunofluorescence, multiplexed antibody-based mass spectrometry (e.g., multiplexed ion beam imaging (MIBI), or cytometry by time-of-flight (CYTOF)), or imaging (e.g., co-detection by indexing (CODEX) imaging), single cell RNA-, protein-, metabolic-, or epigenome profiling, or spatial RNA-, epigenome-, or metabolic profiling. The TIME infiltration value represents a composition of immune infiltrates in the TIME of the tumor of the patient 106.

In some examples, immunohistochemistry, or immunofluorescence, can be used in clinical settings due to low cost relative to other technologies, but other methods can be used to study immune cell infiltrates into tumor to produce the TIME score. In some examples, tumor-specific expanded T cell populations can be determined in the blood, e.g. by T-cell receptor sequencing (TCR) sequencing, enzyme-linked immunospot (ELISpot) sequencing, or tetramer assay).

The TIME of the tumor of the patient 106 refers to the ecosystem surrounding the tumor. The TIME may consist of various cell types, signaling molecules, and extracellular components. TIME infiltration refers to the movement and presence of immune cells within the TIME. Examples of TIME infiltration biomarkers include, but are not limited to, checkpoint expression biomarkers, T-cell infiltration biomarkers, and tertiary lymphoid structure (TLS) signatures biomarkers. The TIME infiltration calculation module 116 is trained on patient RNA expression data which include values indicative of one or more of a checkpoint expression, a T-cell infiltration, and a TLS signature of a set of patients.

The immunotherapy response calculation module 114 can use the TIME infiltration value output from the TIME infiltration calculation module 116 in determining the outputs of the immunotherapy response calculation module 114.

In general, the patient data used to train the immunotherapy response calculation module 114, or the TIME infiltration calculation module 116 can be publicly available patient data, privately available patient data, or independently gathered patient data.

In some examples, the module 114 can be trained using recursive feature elimination (RFE). RFE can be used to identify important features in a set of features to be used in the module 114. In some examples the RFE is an XJBoost model. The RFE may include a nonlinear model for feature selection to allow for feature interactions. The RFE can determine a quality value for each of the features, or combination of features, in the set of features. The RFE may use a mean square error for some, or all, combinations of features in the set of features. Training the module 114 with an RFE can reduce the number of features, or combinations of features, used in the module 114 to determine the ICB response score for the patient 106. Reducing the number of features, or combination of features, can increase the speed of the engine in determining the ICB response score, and can increase the accuracy of the ICB response score for the patient 106.

In some examples, the module 114 can determine a number of mutations presented in an antigen-presenting molecule of the patient 106 using the cellular conditions data received by the engine 110. Determining the number of mutations presented in the antigen presenting molecules of the patient 106 can beneficially increase the accuracy of the determined classification of the patient 106. In some examples the antigen presenting molecule is a major histocompatibility complex class II (MHC-II) or a major histocompatibility complex class I (MHC-I) of the patient 106.

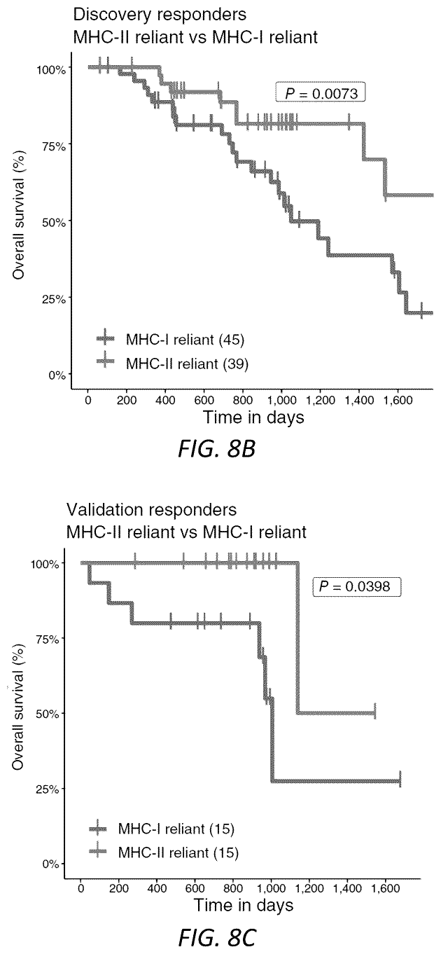

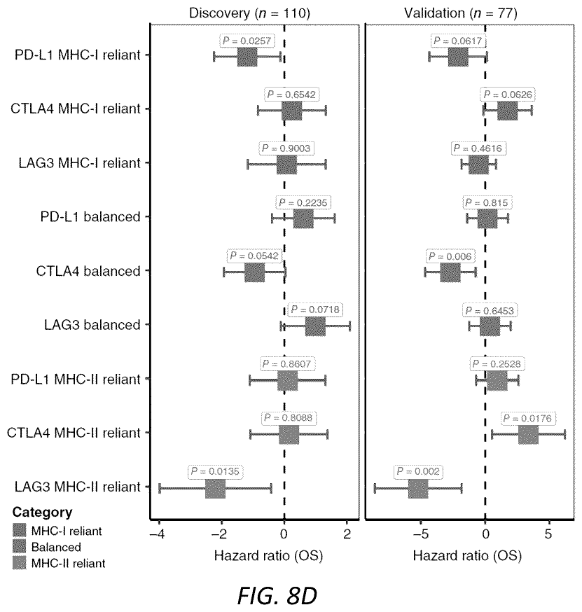

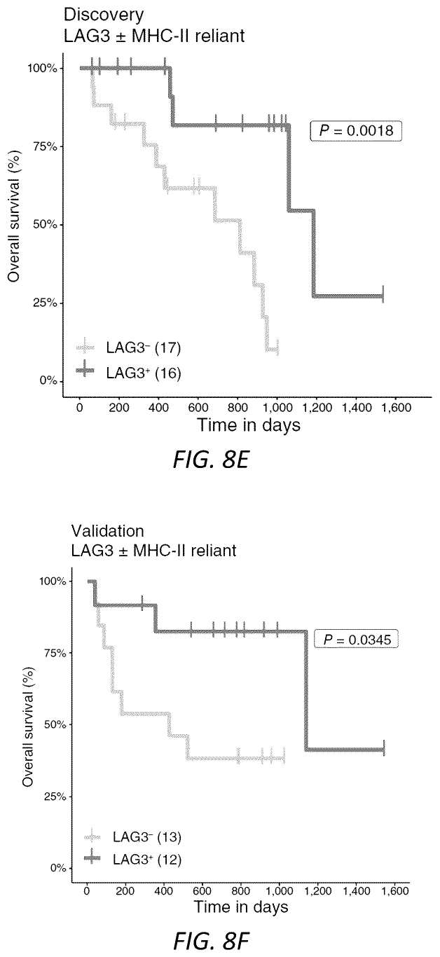

The module 114 uses the determined number of mutations presented in the antigen presenting molecules to calculate a major histocompatibility complex (MHC) ratio of the patient 106. The module 114 determines the MHC ratio of the patient 106 by determining a total number of neoantigens presented by the MHC-II and a total number of neoantigens presented by the MHC-I of the patient 106. The module 114 divides the total number of MHC-II neoantigens by the total number of MHC-I neoantigens to determine the MHC ratio. The module 114, in some examples, uses the MHC ratio in determining the classification of the patient 106. The MHC ratio can be used together with the output of the model to predict increased, e.g., longer-term, benefit among predicted immunotherapy responders. This is shown in FIGS. 8B-8C. The MHC ratio is informative of the correlation between activity of immune checkpoint genes not limited to PD-1, PD-L1, CTLA-4 and LAG3 and response to immunotherapies, shown in FIG. 8D. The MHC ratio module can classify potential to benefit from a combination of immune checkpoint drugs.

In some examples, the feature importance for multiple sequencing features can be calculated to determine the feature vector used by the module 114. Determining the feature importance of the features in the sequencing features can provide a weight for each of the features in calculating the ICB response value.

A number of potential sequencing features to be included in the sequencing feature vector of the module 114 can be compared using one or more comparison models. In some examples the feature comparison model is a Shapley additive explanations (SHAP) model. Multiple sequencing features can be input into the comparison model. The comparison model determines they feature importance value for each of the sequencing features in the multiple sequencing features. The comparison model compares each feature importance value to a feature importance threshold value, e.g., stored in non-transitory memory. If the feature importance value meets the feature important threshold value, the corresponding feature can be included in the sequencing features of the model used by the engine 114.

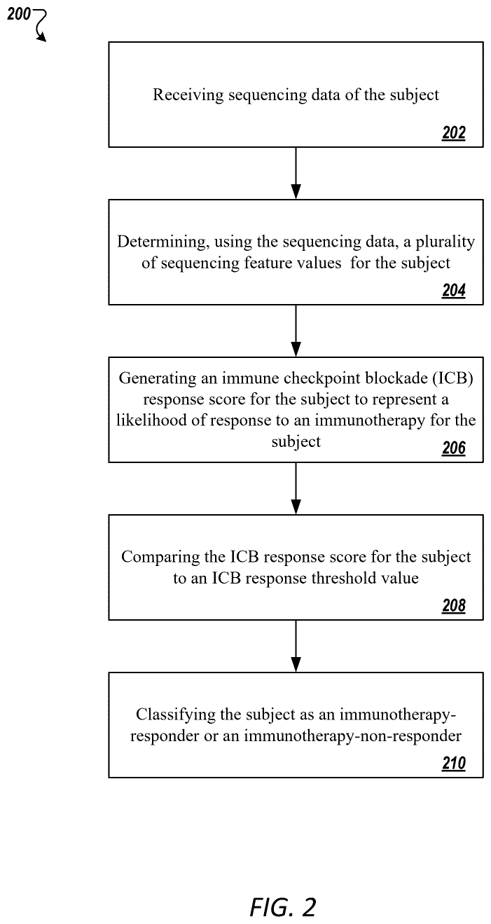

FIG. 2 is a flowchart of an example process 200, e.g., a computer implemented process, for predicting a subject's response to an immunotherapy procedure to treat a cancer for a patient, e.g., patient 106, based on cellular conditions data. In some examples, the immunotherapy procedure is an ICB immunotherapy. The process 200 may be used, for example, by a medical user, e.g., user 102, for a subject's response to an immunotherapy procedure, using an immunotherapy response calculation system, e.g., engine 110.

A system receives cellular conditions data, e.g., sequencing data, of the subject (202). The sequencing data can include DNA sequencing data, genome sequencing data, whole-exome sequencing data, RNA sequencing data, or any combination of these.

The system determines, using the cellular conditions data, sequencing feature values for the subject (204). The sequencing feature values determined by the system include values for the feature vectors which the system was trained to identify as predictive of a patient's response to an immunotherapy. Examples of feature vectors are described here in but can include feature vectors related to somatic features or germline features of the sequencing data of the subject.

The system generates an ICB response score for the subject (206). The ICB response score represents a likelihood of response to an immunotherapy for the subject. Without expressing limitation, the ICB response score is related to germline and somatic biomarker indicator values determined from the sequencing data.

The system compares the ICB response score of the subject to an ICB response threshold value (208). The ICB response threshold value is stored or determined by the system as a value which predicts that the subject is an immunotherapy responder or an immunotherapy non responder. In some examples the system stores the ICB response threshold value in non-transitory memory for comparison. In some examples, the system may be retrained to determine a new ICB response threshold value for future subjects.

The system classifies the subject (210). The system classifies the subject as an immunotherapy responder or an immunotherapy non responder based on the comparison of the ICB response score to the ICB response threshold value. If the ICB response score is greater than the ICB response threshold value, the system classifies the subject as an immunotherapy responder. If the ICB response score is less than the ICB response threshold value, the system classifies the subject as an immunotherapy non responder.

The process 200 may include additional optional steps. Optionally, the system determines an MHC ratio of the subject. The system uses the MHC ratio with the sequencing feature values to classify the subject.

Optionally, the system determines a TIME infiltration value of the subject. The system uses the TIME infiltration value with the sequencing feature values to classify the subject.

The steps of the process 200 need not be performed in the order described above. One or more steps, or optional steps, of the process 200 may come before, or after, other steps of the process 200.

### EXAMPLES

The invention is further described in the following examples, which do not limit the scope of the invention described in the claims.

### Example 1

### Materials and Methods

### ICB and TCGA Data Sets

Raw FASTQ files were obtained using SRA toolkit v2.9.6-1-ubuntu64 for the following immune checkpoint trials: Hugo and colleagues [SRA accession: SRP090294, SRP067938; Cancer: melanoma; n_{1/4}38; RNA sequencing (RNAseq)_{1/4} 27], Van Allen and colleagues (SRA accession: SRP011540, Cancer: melanoma; n_{1/4} 110; RNAseq₄ 40), Miao and colleagues (SRA accession: SRP128156, Cancer: clear cell renal carcinoma; n_{1/4} 69; RNAseq₄ 33), Riaz and colleagues (SRA accession: SRP095809, SRP094781; Cancer: melanoma; n_{1/4} 57; RNAseq_{1/4} 46), Rizvi and colleagues (SRA accession: SRP064805, Cancer: non-small cell lung cancer; n_{1/4} 35), Snyder and colleagues (SRA accession: SRP072934, Cancer: melanoma; n_{1/4} 64), Liu and colleagues (SRA accession: PRJNA82747 Cancer: melanoma; n_{1/4} 122; RNAseq_{1/4} 122), and Cristescu and colleagues (SRA accession: PRJNA449580, Cancer: Melanoma, HSNCC, Urothelial; n_{1/4} 213). Only pretreatment samples were utilized in this study. Across cohorts, a total of 708 ICB-treated patients were evaluated in this study. The Cancer Genome Atlas (TCGA) samples from similar tissues to the ICB cohorts were obtained from the TCGA data portal https://portal.gdc.cancer.gov/. Only patients with available genetic SNP, somatic, and RNAseq data were extracted (n_{1/4} 3,377) from the following TCGA cohorts (LUAD, n_{1/4} 522; KIRC, n_{1/4} 537; SKCM, n_{1/4} 470; HNSC, n_{1/4} 528; BLCA, n_{1/4} 412; KICH, n_{1/4} 113; KIRP, n_{1/4} 291; LUSC, n_{1/4} 504).

### Data Processing

FASTQ files were processed via an identical bioinformatics pipeline. DNA: Genomic reads were aligned to UCSC hg19 coordinates using BWA v0.7.17-r1188. Reads were sorted by SAM-TOOLS v0.1.19, marked for duplicates with Picard Tools v2.12.3 and recalibrated with GATK v3.8-1-0. Germline variants were called from sorted BAM (Binary Alignment Map) files using DeepVariant v0.10.0-gpu. Somatic variants were obtained through the following additional steps. Aligned tumor/normal BAM files were submitted to standard Mutect2 somatic variant calling using GATK-4.1.3.0. First, BAM file formats were standardized using GATK-4.1.3.0 AddorReplaceReadGroups, then GATK-4.1.3.0 Mutect2 was used to call somatic variants using default settings (including the presence of a matched normal), the gnomAD v3.1 raw sites background SNP panel, and the Twist Exome Target bed file to limit variant calling to exonic regions. Potential somatic variants were filtered using GATK-4.1.3.0 FilterMutectCalls and only mutations with a filter flag of “PASS” were kept for subsequent analysis. Somatic mutations were further filtered to retain only those with a DNA allelic fraction 5%. The resulting variant call format (VCF) files were annotated by variant effect prediction using cache version 102_GRCh37 and default settings. RNA: When available, RNA FASTQ/BAM files were downloaded for 33 RCC and 240 patients with melanoma. BAM files were converted to FASTQ using bam2fq. Unpaired reads were removed using fastq pair. Paired reads were aligned with STAR v2.4.1 d to GRCh37 reference alignment. RSEM v1.2.21 was used for transcript quantification. Raw transcript counts were corrected for cohort-specific batch effects using ComBat before being transformed into transcript per million (TPM) values.

### Feature Construction

### Germline Features

A set of 1,084 TIME-associated SNPs was sourced from Pagadala and colleagues. These SNPs were demonstrated in the aforementioned study to have significant associations with immune-related functions in TCGA and were successfully used to develop an earlier germline ICB response prediction model. Next, SNPs present with a mutant allele fraction >0.05 in all studies were filtered for, leaving 598 SNPs to run METAL analysis with ICB response in the three training cohorts. METAL analysis calculates a single P-value for each SNP across the three training cohorts (Hugo and colleagues, Riaz and colleagues, and Snyder and colleagues) and indicates the direction of effect for each cohort. SNPs with an FDR of <0.25 and showing full agreement with direction of impact were included, resulting in 229 SNPs with a nominal ICB association. TCGA and discovery genotype processing was performed by Pagadala and colleagues and is described in detail in their methods. For this study, preprocessed genotype matrices were obtained for each of the cohorts examined.

### Somatic Features

Tumor mutational burden: TMB was defined as the sum of all nonsynonymous somatic coding mutations in each patient's VCF file, including “protein coding,” “frameshift variant,” and “stop lost” mutations. To adjust for cohort-specific effects, TMB was transformed by the intra-cohort z-score before being included in the machine learning model. A similar convention is described in Vokes and colleagues.

Immune escape: A comprehensive list of immune escape-related genes was obtained from Zapata and colleagues. Somatic mutations with variant effect prediction impact annotations of “MODERATE” or “HIGH” were tallied from per patient VCF files. The final immune escape mutation counts were divided by each patient's total TMB to generate a score reflecting disproportionate immune evasion otherwise, the score is highly correlated with TMB.

Antigen presentation pathway: A list of key antigen presentation pathway-related genes was obtained from MSigDB M1062, Reactome Antigen Presentation Folding Assembly, and Peptide Loading of Class-I MHC. All HLA genes were removed from this list as they are accounted for with better accuracy by HLA-specific tools and summarized in other features. Somatic mutations with an impact of “MODERATE” or “HIGH” were tallied from per patient VCF files. The resulting scores were divided by each patient's total TMB to generate a score reflecting disproportionate damage to the antigen presentation pathway.

Intratumoral heterogeneity and fraction of TMB subclonal: Intratumoral heterogeneity (ITH) and fraction of TMB subclonal both rely on accurate subclonal estimates, which are derived as follows. First, copy number calling was performed using CNVkit v0.9.10. A background panel of normals was constructed for each cohort separately using CNVkit reference to protect against batch effects. CNVkit batch was used to call copy number changes with each respective cohort's matched background panel. PureCN v2.6.4 (run via singularity image) was used with CNVkit-derived.cnr and .seg files, and Mutect2-derived filtered VCF files to generate purity and ploidy metrics to be used in subsequent subclone estimation. PureCN was run with default settings, repeat regions censored, and a random seed set to 123. Next, PyClone-VI v0.13.1 was run on mutation-specific integer copy number estimates derived from the CNVkit call (cnvkit.readthedocs.io/en/stable/heterogeneity.html) to estimate the clonal structure of the tumor. ITH was defined as the total number of subclones with at least five mutations (total range 0-11 subclones). Fraction of TMB subclonal was calculated by taking the total number of mutations belonging to small subclones (5 mutations per subclone) and dividing it by the total number of mutations for each tumor. This generates an inverse estimate of clonal heterogeneity from ITH.

Immunoediting: Immunoediting evaluates the ratio of non-synonymous to synonymous mutations (dN/dS) in a tumor as a measure of selection. Immune dN/dS was adapted by Zapata and colleagues in their toolkit SOPRANO (github.com/luisgls/SOPRANO) to calculate the immunoediting score for each patient using an hg19 reference and default settings. Essentially, this score derives from calculating dN/dS across all regions of the proteome predicted to bind the set of patient-specific MHC alleles (i.e., displayed for immune surveillance) and ranges from 0 to −5 with a score above one indicating a higher amount of non-synonymous mutations to synonymous ones.

MHC-I damage: MHC-I damage was defined as the union of POLYSOLVER and LOHHLA results. First, class-I HLA alleles were genotyped via POLYSOLVER [see Patient Harmonicmean Best Rank (PHBR) pipeline methods]. Next, LOHHLA (github.com/mskcc/lohhla), originally published in McGranahan and colleagues, is used to identify copy number losses of HLA alleles. Copy number and purity data are provided to the program and summary statistics about HLA copy number losses are generated. A given HLA allele was marked as lost if the Pval_unique of its loss was 0.05. POLYSOLVER mutation calling [Shukla and colleagues] was used to generate somatic mutation calls of each HLA allele. If an HLA allele was flagged by either of these tools, it was marked as damaged. Alleles were only counted as damaged once even if flagged by both tools. Both programs were provided identical HLA genotypes on a per-patient basis.

### Machine Learning Framework

### Overview

XGBoost classifiers were built for three predictive tasks: ICB response prediction from germline, somatic, and combined features, respectively. Models were fit in two stages: feature selection, followed by model training and evaluation. First, recursive feature elimination (RFE) was conducted on an initial array of features using the Cristescu and colleagues cohort, then trained classifiers to predict ICB response using Hugo and colleagues. (n_{1/4} 34), Riaz and colleagues (n_{1/4} 61), and Snyder (n_{1/4} 64) melanoma cohorts. The trained model was then evaluated on three test cohorts: Vanallen and colleagues (n_{1/4} 110), Miao and colleagues (n_{1/4} 70), and Rizvi and colleagues (n_{1/4} 34). Biological implication validation was conducted with the Liu and colleagues (n_{1/4} 122) cohort.

### RFE

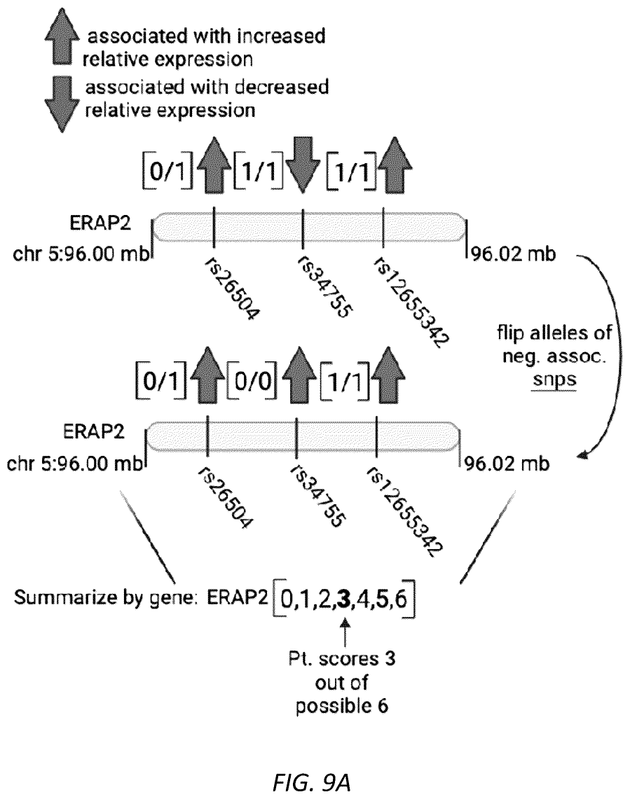

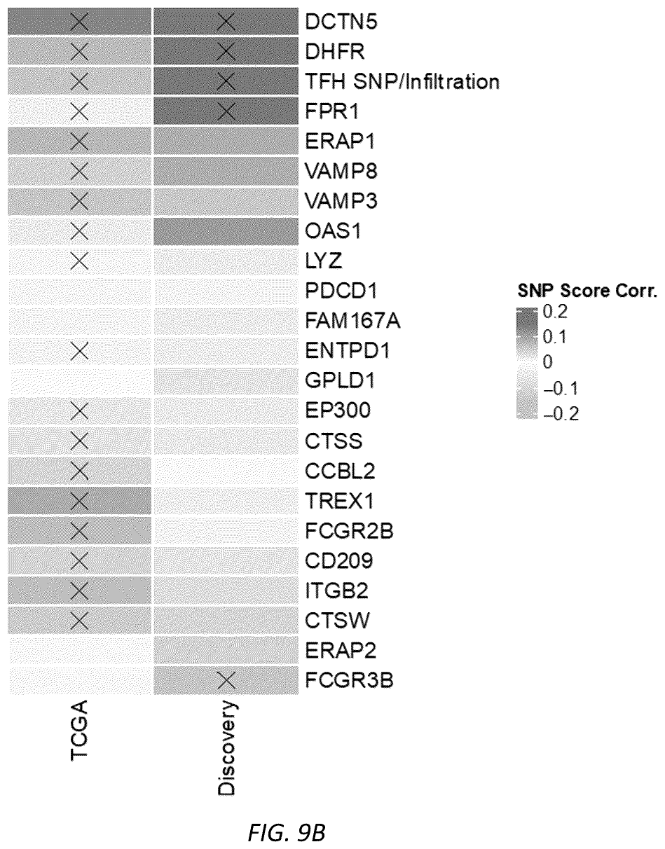

RFE was performed on three feature sets: 229 germline SNPs only, 16 somatic variables only, and both sets combined. The RFE model was trained on Cristescu melanoma (n_{1/4} 89) and tested on Cristescu HNSCC (n_{1/4} 107) and Cristescu urothelial (n_{1/4} 17) samples to ensure this step prioritized broadly useful biological features to use in the model training step. The model used for RFE was an XGBoost random forest classifier (python package version 1.6.2) with 20 total estimators and a maximum depth of 8. a nonlinear model for feature selection was used to allow for feature interactions even during the feature selection stage. Feature combinations and total model sizes were tested, and the mean squared error (MSE) of each was recorded. The model with the lowest MSE was selected, and the features included in that model were used for training in stage two. For the 229 germline SNPs, a model with a combination of 54 SNPs yielded the lowest MSE in the RFE cohorts. These 54 SNPs were collapsed into continuous genelevel expression quantitative trait loci (eQTL) scores by measuring the direction of their effect on gene expression in TCGA (see dataset methods for more details) and orienting alleles such that all SNPs affected gene expression in the same direction (FIG. 9A). This resulted in 23 simplified, gene-level continuous scores reflecting the total magnitude of the expected change in gene expression (FIG. 9B). For the composite model, RFE was performed on the set of features prioritized by the initial RFE performed for each data type separately.

### Icb Response Classifier Training

three different classifiers were trained to predict ICB response, one using only germline features, one using only somatic features, and one on the combined feature set (the composite model). Using features passing RFE analysis, XGBoost random forest classifiers were trained on Riaz and colleagues, Hugo and colleagues, and Snyder data sets with 1,200 total estimators and a maximum depth of 8. The performance of these models was then evaluated separately on the Vanallen and colleagues, Rizvi and colleagues, and Miao and colleagues datasets. Aside from feature curation, this process was identical for all models, and a standard random seed was set for all models to ensure reproducibility. For each patient, the XGBoost random forest classifier returns a class prediction probability ranging from 0 to 1, which were referred to as the immune checkpoint (IC) index. For visualization purposes, sklearn MinMaxScaler was used to scale these values from 0 to 10. This process preserves the distribution of scores and therefore does not affect statistical comparisons.

For each model and cohort, IC index scores were compared between responders and nonresponders using Mann-Whitney U tests. Comparisons between the effect size of each IC index were made using the Cliff's Delta value of each model's effect size. Response to immunotherapy was defined using the RECIST criteria. ROC plots (e.g., sensitivity is plotted on the y-axis against the false positive rate (1−specificity) on the x-axis) were constructed using the scaled continuous IC index results, in which the outcome label was the response phenotype, and the area under the curve (AUC) was used to summarize overall performance. Test datasets were then pooled for survival analysis via multivariable Cox proportional hazard analysis, in which the association of IC index with progression-free survival (PFS) was measured alongside covariates of age, sex, and tumor type, using the R packages “survival” and “survminer” Kaplan-Meier curves were constructed using tertile splits of IC index scores and P-values of pairwise comparisons between tertiles were computed with log-rank tests. Finally, positive, and negative predictive values (PPV and NPV, respectively) were computed and compared between each model type using the “DTComPair” package.

State-of-the-art ICB response prediction projects from Litchfield and colleagues, Chowell and colleagues, and Auslander and colleagues have demonstrated remarkable accuracy in validation sets when RECIST stable disease (SD) category patients are included as nonresponders or excluded entirely. These SD patients are particularly difficult to classify because of their ambiguous TIME and somatic biomarker landscape, but still benefit from increased overall survival and were counted as responders in predictive modeling tasks in this study.

Evaluation of the TIME with Digital Cytometry

The composition of immune infiltrates in the TIME was evaluated by digital cytometry via CIBERSORTx using the LM22 signature matrix with batch correction. The T-cell infiltration score was constructed from the CIBERSORTx CD8 T Cells score. The general TIME score used in Kaplan-Meier plotting was calculated as the linear combination of the therapeutic target, T-cell response, and TLS formation. CIBERSORTx T-follicular helper cell estimates were reused for MHC reliance analyses to corroborate the effect of SNPs associated with higher T-follicular helper cell infiltration. The TLS gene expression signature was generated from a set of TLS-related genes reported by Cabrita and colleagues and Saute's-Fridman and colleagues (CCL19, CCL21, CXCL13, CCR7, CXCR5, SELL, LAMP3, CETP, RBP5, AICDA, BCL6, CCR6, and CD79B; 53) using the method put forth in Cabrita and colleagues, in which mean gene expression of key genes upregulated in TLS was calculated. CD4⁺ and CD8⁺ T-cell infiltration estimates were calculated using CIBERSORTx, in which the CD4/CD8 ratio was defined using “T-cell CD4 memory activated”+“T-cell follicular helper” infiltration divided by “T-cell CD8” infiltration categories. Only patients in the top two tertiles of CD8+ T-cell infiltration were included in direct CD4/CD8 ratio comparison analysis to remove patients with zero or very low levels of immune infiltrates.

### Shap Feature Importance and Feature Interactions

Feature importance and interaction within nonlinear models were calculated using the SHapley Additive exPlanations (SHAP) machine learning interpretability suite (shap.readthedocs.io/en/latest/). SHAP is a unified approach to explain the output of any machine learning model. It is based on cooperative game theory and the concept of Shapley values. SHAP values assign each feature an importance value for a particular prediction in the context of a specific model. These values allow for nonlinear interactions between features to be accounted for on a per-patient basis, and they also allow us to rank pairwise feature interaction by magnitude. Each model was run through the standard SHAP python pipeline and the feature importances were recorded. For the composite model, feature interaction analysis was performed as well using the shap_interaction_values function.

### Phbr Score Pipeline

Originally developed by Marty and colleagues the PHBR score is a measure of how well a given neoantigen is presented by the MHC based on computationally derived binding affinities between peptides harboring the mutation and a patient's set of HLA alleles. A detailed description can be found in the original publication. For each patient, all single nucleotide variant mutations were given an MHC-I PHBR score and an MHC-II PHBR score representing presentation by class-I and class-II, respectively. A neoantigen was considered to be well presented by MHC-I with a PHBR score ≤2 and well presented by MHC-II with a PHBR score ≤10. Class-I HLA alleles were called using POLYSOLVER (v1.0.0) with default parameters, and Class-II HLA alleles were called using HLA-HD (v1.4.0) with default parameters.

### Mhc Reliance Stratification

Patients were stratified by the ratio of the total number of neo-antigens well presented by MHC-II divided by the total number of neoantigens well presented by MHC-I. A patient was only considered for analysis if they had at least three mutations well presented by both MHC-I and MHC-II. Neoantigens that were both well presented by both MHC-I and MHC-II were not considered in this ratio. These ratios were divided into tertiles and defined as follows: the lowest tertile was MHC-I-reliant, the middle tertile was balanced, and the highest tertile was MHC-II reliant. To select for patients with MHC II-based immune responses, MHC II-reliant patients with no evidence of MHC-I damage or loss of heterozygosity were excluded.

### Tcga Immune Infiltration Analysis

Tissue types matching those from the analysis (melanoma, renal cell carcinoma, non-small cell lung carcinoma, head and neck squamous cell carcinoma, and urothelial/bladder cancer) were pulled from TCGA: LUAD, KIRC, SKCM, HNSC, BLCA, KICH, KIRP, and LUSC (see ICB and TCGA datasets). Stage II-IV cancers were analyzed to better match the ICB cohorts. Poorly infiltrated tumors were dropped from the analysis to ensure that cancers analyzed from TCGA were at least somewhat infiltrated by lymphocytes. To achieve this, the ImmunoScore, for all patients, was calculated and the bottom tertile (most poorly infiltrated) patients were dropped from the analysis. CD4/CD8 T-cell ratios were calculated in an identical manner as the ICB cohorts. Similarly, MHC reliance groupings were generated identically as in ICB discovery and reliance cohorts.

### Tcga Tumor Intrinsic MHC-II Expression Estimates

Tumor intrinsic MHC-II expression was estimated by adjusting HLA DRB1 expression in the same cancer types and stages as the above section (see “TCGA immune infiltration analysis”). HLA DRB1 expression levels were corrected for inter-patient variation in immune infiltrates by multiplying by tumor purity fraction and 1 minus the sum of relative infiltration of professional antigen-presenting cells and CD4⁺ T cells, as measured by CIBERSORTx. The following canonical MHC-II expressing cell types in the CIBERSORTx LM22 matrix were adjusted for: B cells, CD4⁺ T cells, macrophages, and dendritic cells.

### Multivariable Checkpoint Analysis

Five FDA-unapproved IC genes with ongoing clinical trials were investigated for an association with a particular MHC reliance group: LAG3, TIM3, TIGIT, OX40, and IDOL. Univariable analysis revealed significant associations with LAG3 in both discovery and validation ICB cohorts, which were subjected to further multivariable analysis accounting for PDL1 and CTLA4 expression. A median expression cutoff was used to create binary high- and low-expressing groups for each of the checkpoint genes. Age, sex, and tumor type were accounted for during multivariable Cox proportional hazard analysis, as well as prior CTLA4 treatment in the validation cohort, because of a large proportion of patients in Liu and colleagues having received such treatment. Kaplan-Meier curves were generated using these same binary cutoffs and P-values were calculated using the log-rank test.

### Statistical Analyses

Statistical software used in this manuscript were R version 4.2.1 and Python version 3.9.2. Unless otherwise indicated all P-value significance thresholds were set at <0.05. Where indicated, P-values were corrected using the Benjamini-Hochberg method.

### Data Availability

This study relied entirely on de-identified publicly available datasets and does not necessitate IRB review. The studies relied upon in this manuscript were all conducted in accordance with recognized ethical guidelines and approved by an institutional review board. Code Availability Code to reproduce models, analyses, and figures can be found at the following Github repository github.com/cartercompbio/MHC_reliance. The relevant Code Ocean capsule can be found here: codeocean.com/capsule/9714470/tree/v1.

### Results

### Design and Evaluation of a Machine Learning Framework to Predict ICB Response

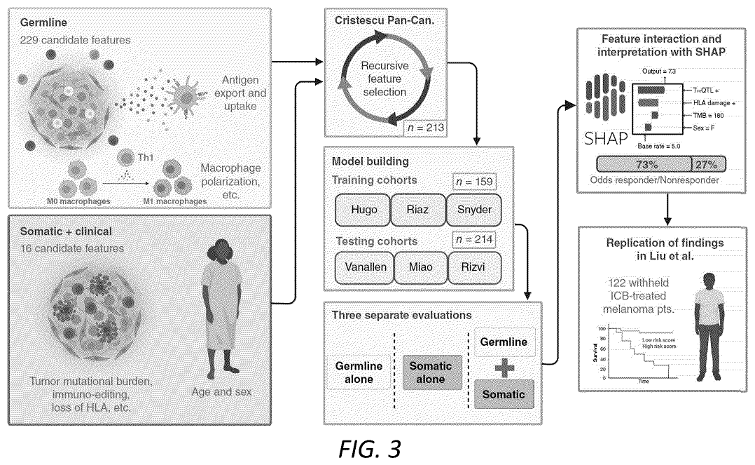

Paired tumor/normal WES data were obtained for eight independent ICB studies encompassing a range of tissue types and treatments across a total of 708 patients. Seven of these were used for machine learning, including feature selection, model training, and independent validation (FIG. 3).

FIG. 3 is flowchart diagram illustrating a combination of germline and somatic/clinical features into a machine learning framework. Germline and somatic features were assembled from the literature and computed from WES data for seven ICB studies (n ¼ 708). Prior to training ICB response predictive models, features and clinical covariates were subjected to RFE using the Cristescu study cohort. Models predicting ICB response were then trained on the selected features using three combined studies as the training set. Performance of the trained model was evaluated separately in three independent cohorts. Models trained only on germline features, only on somatic features, and on the combination were compared. Feature contributions to the trained model were further investigated to develop biological hypotheses that were reproduced in the Liu cohort. Pan-Can, pan-cancer cohort; pts, patients; Th1, T helper 1 cell.

The eighth was added later to validate the translational potential of biological findings. A set of germline and somatic features were assembled that could be extracted from WES data and that have previously been reported to predict ICB response. Germline SNPs associated with the TIME and ICB response from Pagadala and colleagues were further harmonized and aggregated at the gene level into numerical scores for their respective gene, here termed eQTL-scores.

Referring briefly to FIGS. 9A and 9B, FIG. 9A schematically illustrates a method for determining an eQTL-score. SNPs alleles were oriented to be in alignment in terms of direction of effects on gene expression. The number of alleles affecting gene expression were summed into a continuous score.

FIG. 9B illustrates a table showing Pearson correlation of Gene level eQTL-scores in the TCGA and Discovery cohorts. P<=0.05 is indicated by an X.

Referring again to FIG. 3, SNPs associated with immune infiltration levels were encoded at the single SNP level instead. Somatic features from several impactful ICB response prediction studies were generated for each cohort, including TMB, dN/dS of the immunopeptidome (immunoediting), damage of MHC-I alleles and somatic mutation of genes in the antigen presentation pathway. Clinical features available for all data sets included patient age and sex. To train models to predict ICB response, a twostage machine learning approach was used entailing feature selection followed by model training (FIG. 3). The number of features were reduced via RFE using the Cristescu and colleagues cohort before training an XGBoost classifier to predict ICB response as class labels. XGBoost is a tree-based ensemble method that generates a continuous probability score, here scaled to range between 0 and 10. Three similar anti-PD1/anti-PDL1/anti-CTLA4 treated melanoma cohorts were combined [Hugo and colleagues, Riaz and colleagues, and Snyder and colleagues] into a single training set, and evaluated the potential of the classifier to generalize by applying it separately to three heterogeneous independent test cohorts: Van Allen (anti-CTLA4 treated melanoma), Rizvi (anti-PD1 treated NSCLC), and Miao [anti-PD1 or anti-PDL1 some also with anti-CTLA4 treated RCC]. Models that relied only on germline features, only on somatic features, or on a combination of both (referred to as the composite model) were compared. The scores produced by these models were termed the IC index.

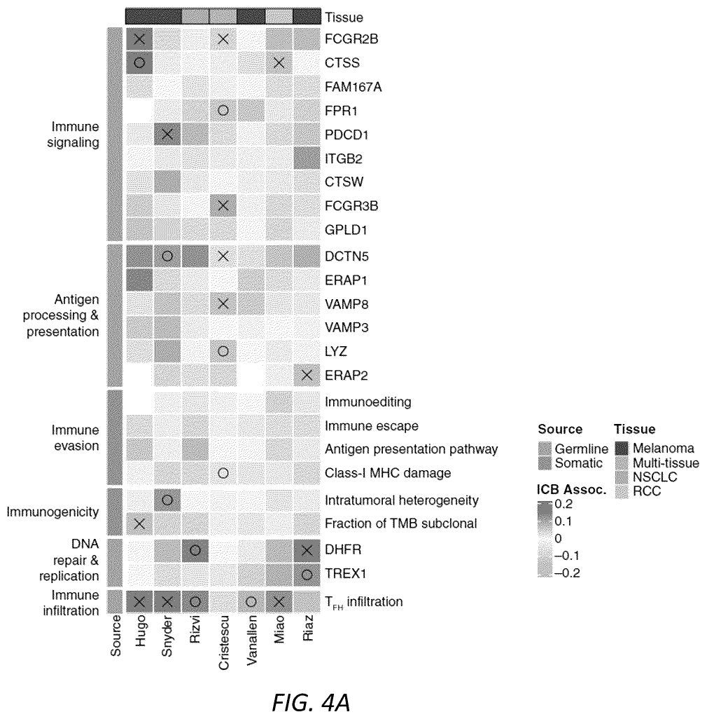

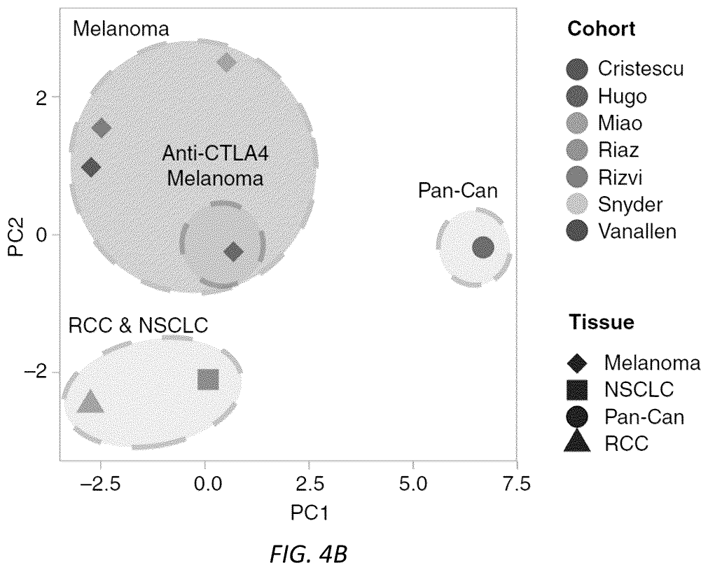

After RFE, 24 germline features were retained to train the germline model, including 23 germline eQTL-scores representing genes involved in antigen processing/presentation [ERAP2, ERAP1, VAMP8], immune signaling [FCGR2B, PDCD1, CTSS, CTSW], and DNA replication [DHFR, TREX1] and a SNP associated with infiltration of T-follicular helper cells (T_{FH}QTL), which was strongly and consistently associated with response across all cohorts (FIG. 4A).

FIG. 4A is a matrix chart comparing features from germline and somatic models passing secondary RFE on the Cristescu pan-cancer cohort are shown grouped by cohort and biological impact. Coefficients quantifying associations with ICB response calculated from generalized linear model analysis accounting for age and sex are shown. Green coloring indicates a feature that was more associated with a positive ICB response, yellow indicates an association with a negative response. Associations with P≤0.05 are marked with an X, P≤0.1 are marked with O.

RFE for the somatic-only model selected 13 features derived from clinical and tumor genomic data, including TMB, clonality-aware derivatives of TMB such as ITH, and fraction of TMB subclonal as well as DNA based T-cell infiltration estimates and measures of immune evasion (immunoediting, immune escape, MHC-I damage, and antigen presentation pathway damage). RFE for the composite model selected 24 features, 18 (75%) of which were germline eQTL-scores and six (25%) of which were somatic features. Considered independently, only a minority of these features showed a significant association with ICB response, and although the direction of effects generally agreed, there was variability across datasets (FIG. 4A).

Feature associations with ICB response were more similar across melanoma cohorts than other tumor types (FIG. 4B). FIG. 4B is a PCA chart of test statistics for each cohort resulting from linear feature associations. The abbreviated terms of FIG. 4B are used to mean Assoc.: association; NSCLC: non-small cell lung cancer; Pan-Can: pan-cancer cohort; and RCC: renal cell carcinoma.

Although TMB and clonal TMB features passed RFE in the somatic-only model they were eliminated in the composite model, which instead utilized fraction of TMB subclonal and ITH features that are anticorrelated and correlated with TMB, respectively (fraction of TMB subclonal: R=−0.22, P=5.9e⁻⁰⁸; ITH: R=0.2, P=1.6e⁻⁰⁶). The composite IC (cIC) index produced by the trained model remained somewhat correlated with TMB (R=0.2; P=0.0035) even though TMB was not directly incorporated as a feature. The somatic IC index had a high correlation with TMB (R=0.46; P=7.5e⁻¹³), whereas the germline IC index was completely uncorrelated with TMB (R=−0.059; P=0.39). Finally, purity and ploidy were somewhat correlated in the model (R=0.18; P=4e−06).

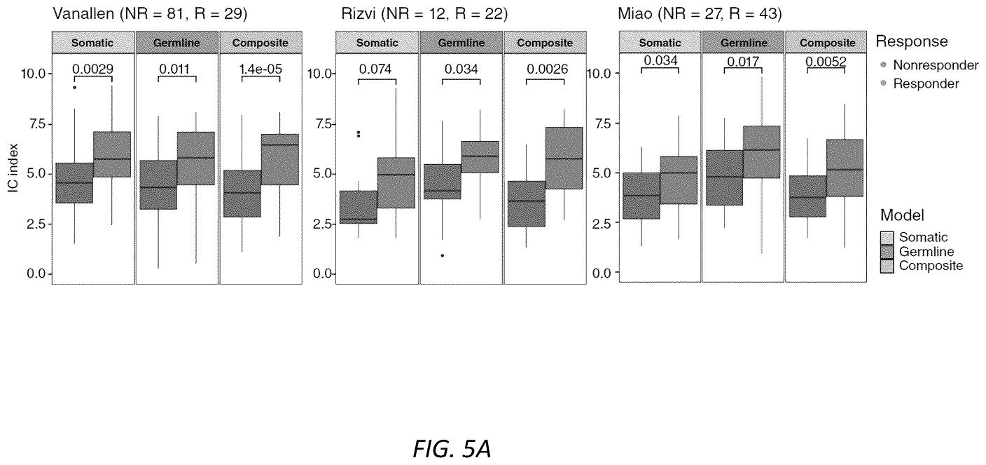

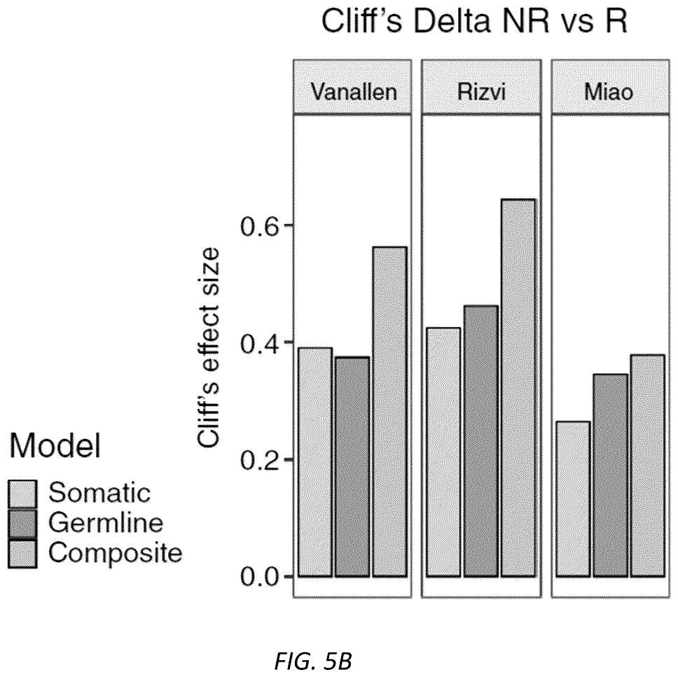

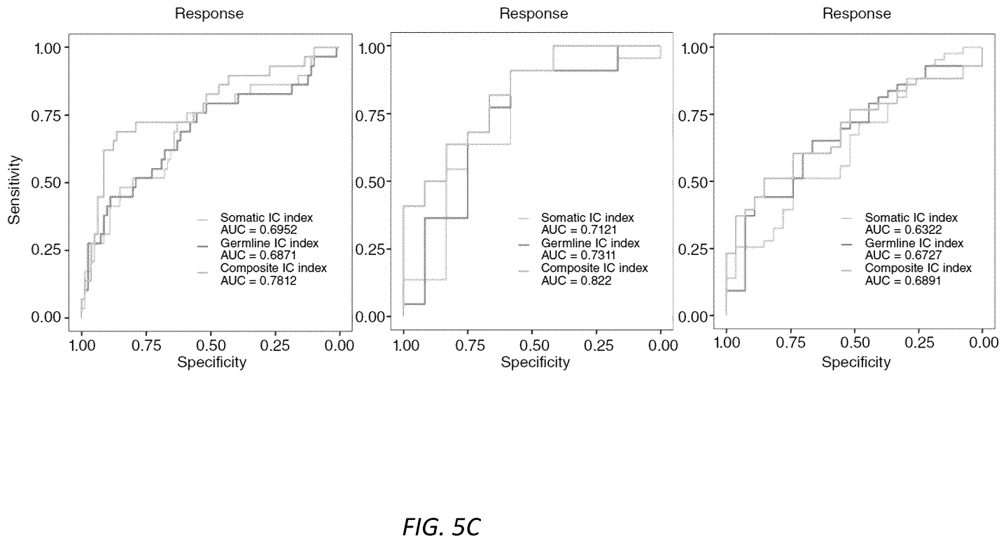

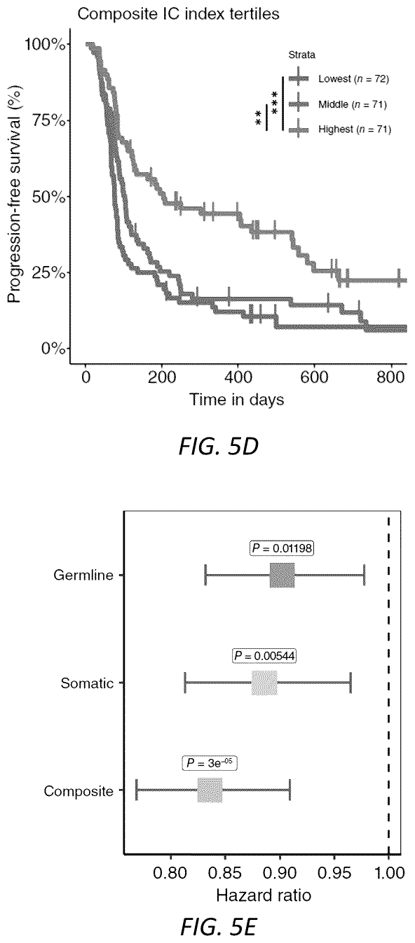

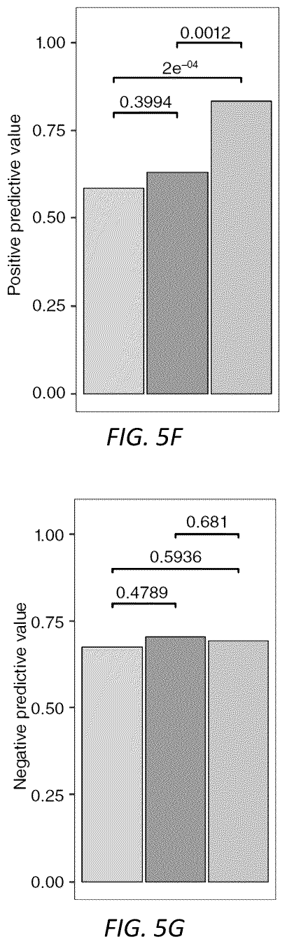

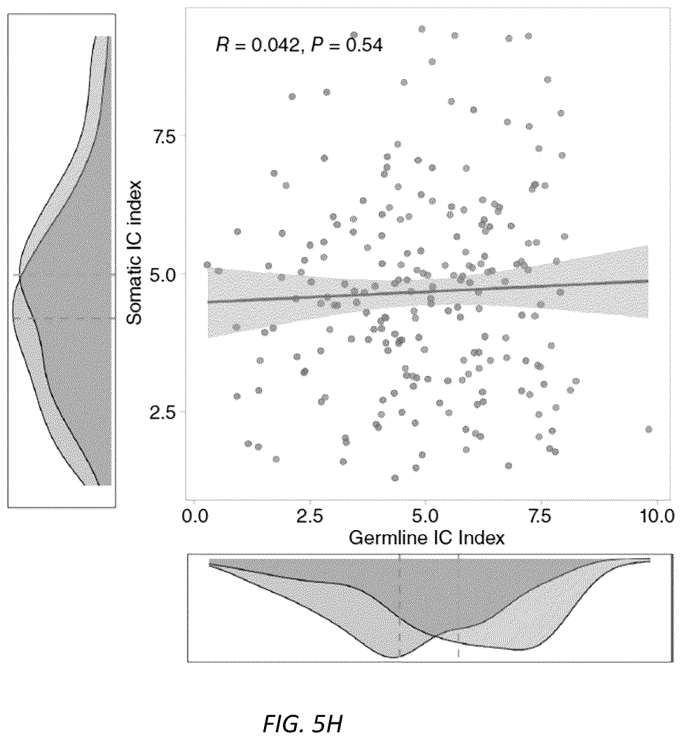

After training XGBoost models on the selected features using the combined training set, the performance of each model was compared on the three independent test sets. Although all three models could distinguish between responders and nonresponders, the cIC index showed the best performance, resulting in the largest mean shift in score distributions between responders and nonresponders (FIG. 5A), the highest Cliff's Delta between responders and nonresponders (FIG. 5B), and the highest area under the ROC AUC (FIG. 5C).

FIGS. 5A-5C show an evaluation of germline, somatic, and composite models across testing cohorts. FIG. 5A illustrates boxplots of medians of testing cohorts in which IC index scores are compared via Mann-Whitney U tests. Error bars represent standard deviation. FIG. 5B illustrates the effect size (Cliff's Delta) between IC index of responders vs. nonresponders of each model and cohort. FIG. 5C illustrates the ROC curves of each model's performance across testing cohorts with AUC.

Improvements in ROC AUC from approximately 0.7 to 0.8 were observed in the Van Allen and Rizvi studies, but more modest improvements were observed in Miao, possibly due to the vastly different TIME landscape of renal cell carcinomas compared with melanomas. PFS of the highest tertile of cIC index scores was significantly higher than the lowest tertile in Kaplan-Meier analysis (FIG. 5D, P >0.0001) and the cIC index was more predictive of PFS in a Cox proportional hazard analysis using age, sex, and tumor type as covariates (see “Materials and Methods”), with a more extreme hazard ratio (HR) and more significant P-value relative to germline and somatic-only models (FIG. 5E). FIG. 5D illustrates the Kaplan-Meier plots of composite IC index score tertiles. FIG. 5E illustrates the HR of IC index for each model from a multivariable Cox proportional hazard analysis of PFS across aggregated testing cohorts accounting for age, sex, and cohort.

Compared with germline and somatic-only models, the cIC index resulted in an increased PPV (FIG. 5F; P_{1/4} 0.0012; P_{1/4} 2e⁻⁰⁴), whereas NPV was not significantly different (FIG. 5G). FIG. 5F illustrates the positive predictive value of each model aggregated across all cohorts. FIG. 5G illustrates the negative predictive value of each model aggregated across all cohorts.

In addition, it was found that the germline IC index and somatic IC index scores were completely uncorrelated with each other, suggesting that these sources of data capture orthogonal information (FIG. 5H; R_{1/4} 0.042; P_{1/4} 0.54), helping to explain the improved performance of the composite model. FIG. 5H illustrates the correlation of germline IC index and somatic IC index, with somatic and germline model density plots of IC index for responders (R) vs. nonresponders (NR).

The cIC index also outperformed baseline ICB response predictors including TMB, age, gender, and checkpoint expression. On a pan-cohort basis, the difference in the cIC index of all responders versus all nonresponders was very significant (P_{1/4} 7e⁻⁰⁹).

### Impact of TIME on ICB Response Prediction

Next, the cIC index were compared to characteristics of the TIME that can be obtained from RNA sequencing data, which were available for 34% of test set patients. Several such measures, including effector CD8+ T-cell infiltrates, joint B and CD4+ T-cell levels potentially indicative of TLS formation, and target checkpoint expression (PDL1/CTLA4), have been previously correlated with ICB response.

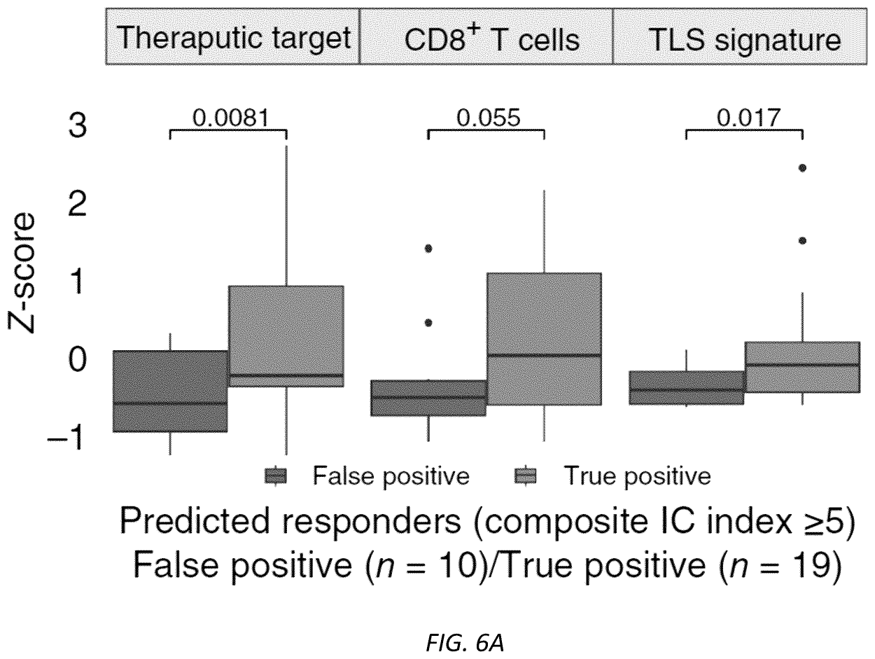

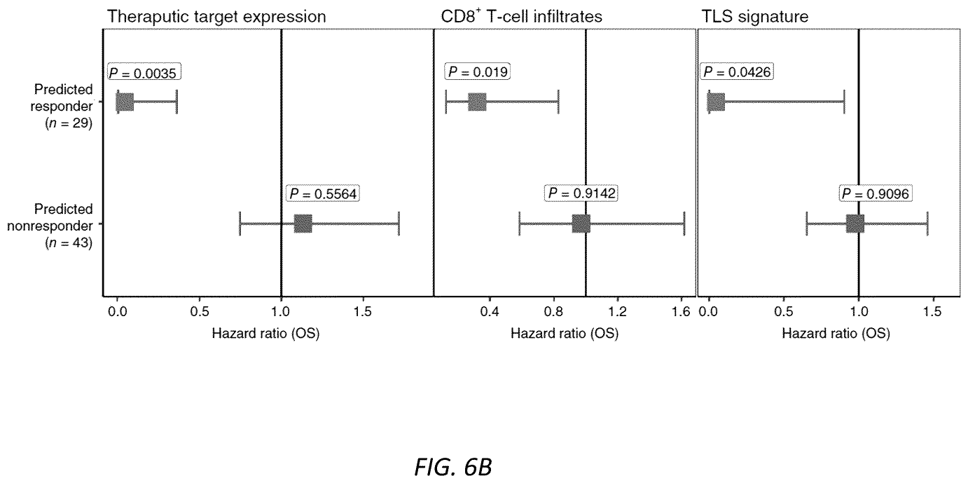

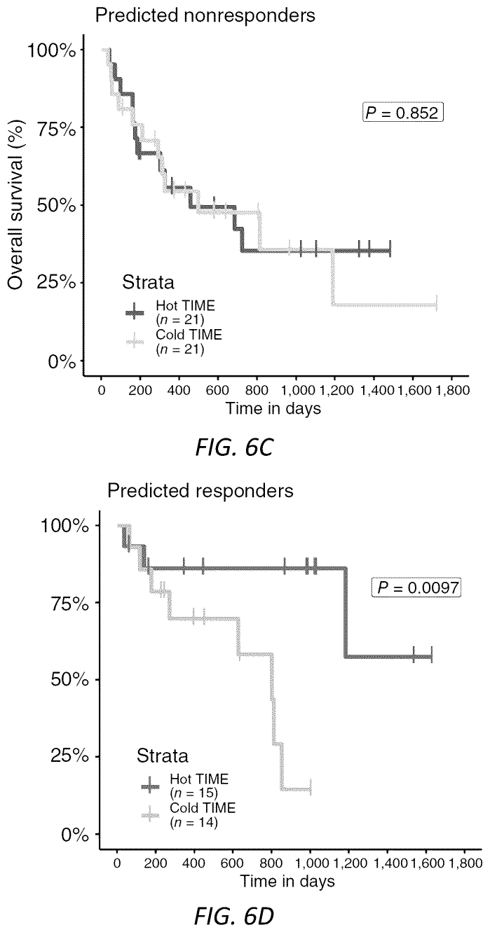

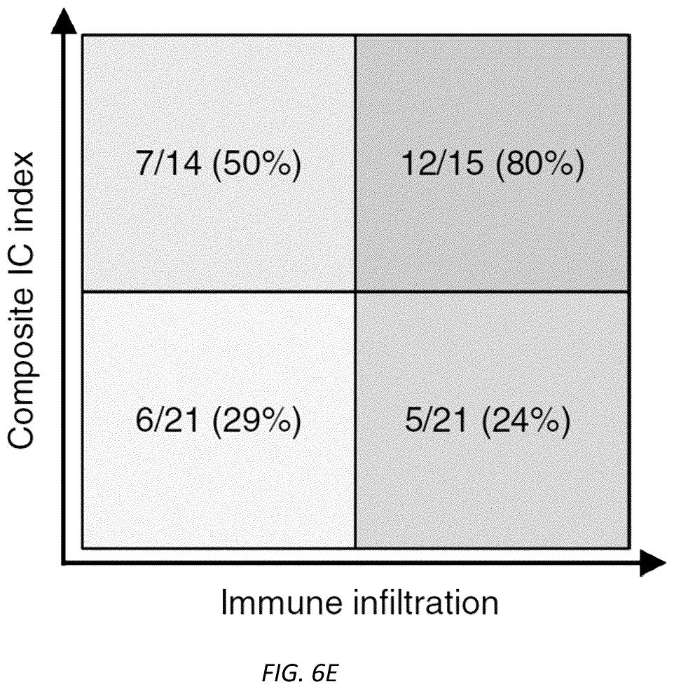

FIGS. 6A-6E illustrate an immune-infiltrated TIME and high cIC index scores are synergistic. CD8+ T-cell infiltration levels with were evaluated CIBERSORTx, a digital cytometry tool that estimates immune cell fractions. To model TLS, the gene signature developed by Cabrita and colleagues was used as a proxy for TLS formation. It was found that patients split by high versus low cIC index (≥5) generally had similar TIME infiltration levels in all three categories. Conversely, the TIME was significantly different between true positives and false positives, in which patients who were predicted to respond (cIC index ≥5) failed to respond and often had an immune-cold TIME, characterized by lower overall levels of immune infiltrates (FIG. 6A). FIG. 6A illustrates boxplots of median expression-based immune measures from combined test samples with RNA sequencing. IC index score distributions are compared with Mann-Whitney U tests. Error bars represent standard deviation.

This relationship was strongest in the checkpoint therapy target (CTLA4 for Van Allen and colleagues, PDL1 for Miao and colleagues; P_{1/4} 0.0081) and TLS formation TIME categories (TLS gene signature P_{1/4} 0.017), with CD8⁺T cells showing near significant association (P_{1/4} 0.055). These results imply that high cIC index patients with favorable germline and somatic biomarkers can nonetheless fail to respond to ICB due to a poorly infiltrated TIME. Whether an immune hot TIME could rescue patients with low somatic and germline potential was investigated for response. Using a Cox proportional hazard model adjusted for age, sex, and data set, it was found that each of the TIME infiltration estimates (checkpoint target: P_{1/4} 0.0035, CD8⁺ T cells: P_{1/4} 0.019, TLS formation: P_{1/4} 0.043) was significantly associated with improved overall survival in high cIC index patients only, whereas low IC index patients failed to significantly benefit from an immune hot TIME (FIG. 6B). FIG. 6B illustrates the hazard plots of primary TIME biomarkers stratified by IC index score. Error bars represent 95% CI.

These results were mirrored in Kaplan-Meier plots of high and low cIC index patients (FIGS. 6C and D) stratified by level of TIME infiltration. FIG. 6C illustrates the Kaplan-Meier curves of predicted nonresponders (IC index <5) stratified by TIME biomarker score. FIG. 6D illustrates the Kaplan-Meier curves of predicted responders (IC index ≥5) stratified by TIME biomarker score. High cIC index patients benefit from an above median TIME (P_{1/4} 0.0097), whereas low cIC index patients do not (P_{1/4} 0.852). Similarly, a high cIC index with an immune hot TIME had the highest rate of response to ICB (FIG. 6E). FIG. 6E illustrates the confusion matrix of IC index (cutoff IC index ≥5) and TIME score (cutoff TIME score above median). The abbreviated term OS represents ‘overall survival.’

These findings are consistent with previous studies indicating that immunogenic tumors respond at greater rates when there is high CD8⁺ T-cell infiltration but that high CD8⁺ T-cell infiltration alone is not sufficient for high rates of ICB response. Furthermore, although high cIC index scores yielded the strongest relationship with higher immune infiltration, it was found that this enhancement was primarily driven by germline factors rather than somatic. Our analyses suggest that cIC index scores may be useful as general estimates of immunogenicity and could be used as additional indicators of when a patient could benefit from ICB beyond TIME profiling.

### Nonlinear Feature Interactions Reveal Alternative Mechanisms of ICB Response

In order to better understand how selected germline and somatic features contribute to model performance, feature importance was analyzed using SHAP values, a game theory approach to improve the interpretation of the machine learning model. Differences were noted in feature rankings particularly for ERAP1, MHC-I damage, and immunoediting, between XGBoost and linear models suggesting the presence of interactivity effects. Thus, both individual feature contributions and pairwise interactions between features were evaluated.

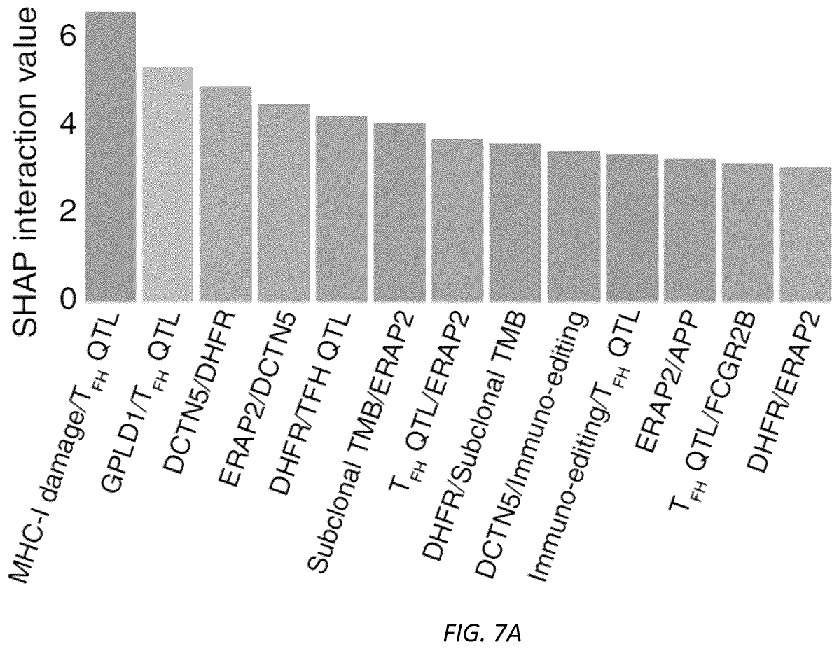

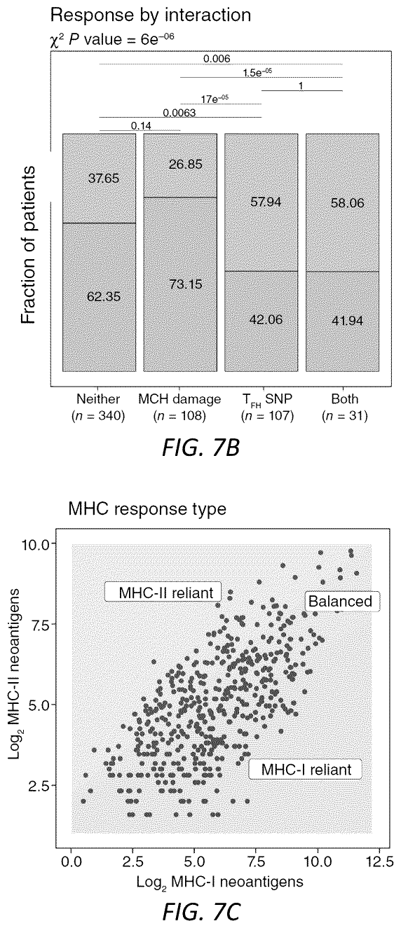

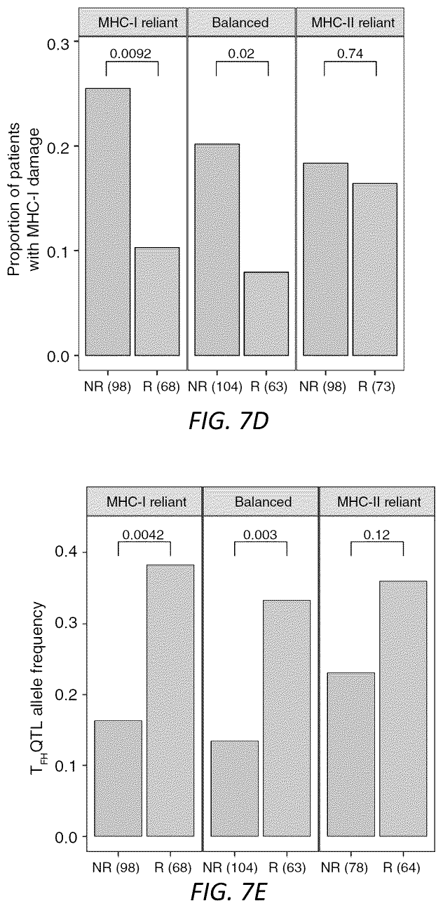

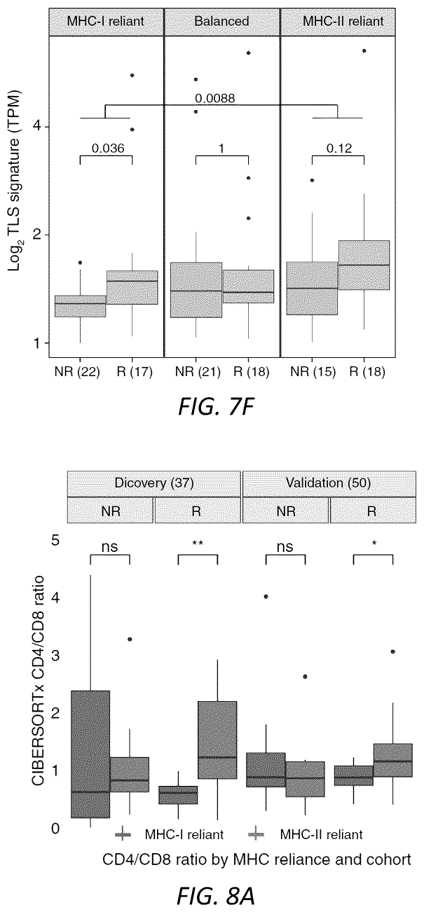

FIGS. 7A-7F illustrate a nonlinear feature interaction analysis reveals differences in response mechanisms. SHAP analysis revealed several key feature interactions (FIG. 7A), the strongest of which was between somatic MHC-I damage, i.e., the cumulative MHC-I damage from somatic mutation and loss of heterozygosity (see “Materials and Methods”), and T_{FH}QTL. FIG. 7A illustrates the SHAP interaction scores for the (top) 12 interacting feature pairs.

This interaction was examined in terms of ICB response rates between categories (FIG. 7B) and observed higher rates of response when the T_{FH}QTL was present (P_{1/4} 1.7e⁰⁵ T_{FH}QTL vs. class-I MHC damage; P_{1/4} 0.0063 T_{FH}QTL vs. neither), even when the potentially negative effect of MHC-I damage was present (P_{1/4} 1.0; T_{FH}QTL vs. both). Because rates of ICB response were unaffected by MHC-I damage in patients carrying the T_{FH}QTL (FIG. 7B), it was hypothesized that this SNP may promote immune responses upon ICB treatment that do not rely on MHC-I-based antigen presentation. FIG. 7B illustrates the bar plot of percent responder by interaction category for the MHC damage and TFH SNP interaction category.

Instead, patients with the T_{FH}QTL could be predisposed toward an MHC-II-driven mechanism of response. As T_{FH} cells primarily assist B cells in producing antibodies through MHC-II interactions, this may suggest a role for humoral immunity that is independent of cellular immune responses mediated via MHC-I and cytotoxic T cells. NK cells are also known to modulate adaptive immune responses via CD27 in MHC-I-deficient tumors. Although the role of follicular T helper cells (Tfh) in this context is not well understood, one study in mice with MHC-I-deficient tumors found that NK cell-CD4⁺T-cell interplay led to tumor rejection without any CD8⁺ T-cell activity.

To further investigate this idea, tumors in the dataset were grouped according to whether somatic mutations were more prevalently presented by MHC-I or MHC-II molecules, suggesting the potential for the reliance of immune responses on particular MHC pathways of neoantigen presentation. First, PHBR scores were calculated for each nonsynonymous mutation in all patients. PHBR scores are mutation-centric scores that seek to summarize whether any peptides overlapping the mutated site will be presented by any of an individual's HLA alleles. Patients with at least three mutations passing PHBR thresholds for both MHC-I and MHC-II were then split into groups termed MHC I reliant, MHC II reliant, or balanced based on the ratio of these class-specific neoantigens (FIG. 7C), with reliant referring to an immune response potentially dependent on MHC-I versus MHC-II presented neoantigens. FIG. 7C illustrates the schematic of MHC-based response groupings.

Among MHC I-reliant patients, it was noted that a significantly higher level of MHC-I damage in nonresponders versus responders (P_{1/4} 0.0092; FIG. 7D) reflecting the notion that an MHC I-reliant response depends on the integrity of the MHC-I and associated antigen presentation pathway. FIG. 7D illustrates the proportion of patients with MHC-I damage split by MHC response categories and response status. Although balanced patients demonstrated an intermediate disparity in MHC-I damage between nonresponders versus responders (P_{1/4} 0.02), this was not the case in MHC II-reliant patients (P_{1/4} 0.74). Overall ICB response rates between these two groups were not significantly different.

Next, it was sought to understand how MHC reliance could modify the potential to benefit from the T_{FH}QTL. It was reasoned that the most extreme cases of MHC-II reliance would be those that also had defects in the MHC-I antigen presentation pathway. The distribution of defects to the MHC-I antigen presentation pathway was statistically similar between each of the MHC reliance groups, although MHC II-reliant patients with defects to the MHC-I antigen presentation pathway showed significantly less immunoediting than those with defects, suggesting that these defects may limit a patient's ability to mount an MHC-I driven immune response. MHC II-reliant patients with defects to the MHC-I antigen presentation pathway comprised 83% of MHC II-reliant tumors, so further analyses on this subpopulation was focused. A significant difference was found in the frequency of the TFHQTL between responders versus nonresponders in the MHC I-reliant and balanced categories (P_{1/4} 0.0042; P_{1/4} 0.003; FIG. 7E) but not in the solely MHC II-reliant category (P_{1/4} 0.12). FIG. 7E illustrates the allelic fraction of the TFHQTL faceted by MHC response categories and split by response status.

This is somewhat mirrored in the subset of patients with tumor immune infiltration estimates available, in which T_{FH} cell estimates were higher in MHC I-reliant responders versus nonresponders (P_{1/4} 0.03) but not in the balanced or MHC II-reliant responders versus nonresponders (P_{1/4} 0.48, P_{1/4} 0.5). It is possible that MHC I-reliant responders benefit from an increased infiltration by T_{FH} cells, TLS formation, and associated helper effects that are important to maintain the function and precursor frequency of CD8⁺T cells. Indeed, TLSs have been shown to enhance ICB response in melanoma Conversely, MHC II-reliant patients may receive less benefit from additional T_{FH}-cell infiltration because their neoantigen landscape is already predisposed toward the formation of TLSs. Indeed, it was found that MHC I-reliant responders had higher TLS gene signature expression than nonresponders (P_{1/4} 0.036; FIG. 7F), yet this difference was not significant in MHC II-reliant patients (P_{1/4} 0.12; FIG. 7F). MHC II-reliant patients in general had a higher level of TLS gene signature expression than MHC I-reliant patients (P_{1/4} 0.0088; FIG. 7F), which is consistent with the fact that TLS formation is more closely associated with the MHC-II/CD4⁺T-cell axis. FIG. 7F illustrates the boxplot of median TLS signature expression split by MHC response categories and response status. Error bars represent standard deviation. Abbreviations include NR: nonresponders; R, responders; and TPM, transcript per million.

These initial observations point to the possibility that mechanistically divergent immune responses yield ICB responses based on how effectively neoantigens engage each MHC pathway.

MHC reliance groupings are related to survival and mechanism of immune evasion To validate the findings, identical analyses were performed on an additional independent ICB-treated cohort (n_{1/4} 77) with paired transcriptomic data [Liu and colleagues] and compared the results with those from the original set of seven cohorts referred to as the discovery set. The effects of MHC reliance grouping was investigated on the composition of the TIME. FIGS. 8A-8F illustrate a TIME, survival duration, and checkpoint expression impact differing by MHC reliance status.

It was observed that CD4⁺/CD8⁺ T-cell ratios mirrored MHC reliance in responders, with higher ratios being observed in MHC II-reliant tumors (FIG. 8A; P_{1/4} 0.0057 discovery; P_{1/4} 0.025 validation). FIG. 8A illustrates a CIBERSORTx CD4/CD8 T-cell infiltration estimate ratios split by MHC reliance category and response status for both discovery and validation cohorts.

However, no such difference was found in nonresponders. An identical methodology to immune-infiltrated ICB-naive, tissue-matched cancer samples from TCGA were applied and a protective effect of the CD4⁺/CD8⁺ T-cell ratio in TCGA MHC II-reliant patients was found (HR_{1/4} 0.76; P_{1/4} 0.0069) but a an adverse effect of that same ratio in TCGA MHC I-reliant patients (HR_{1/4} 0.59; P_{1/4} 0.0352). Additionally, an estimate of tumor intrinsic MHC-II expression was found to be protective in MHC II-reliant patients (HR_{1/4} 0.64; P_{1/4} 0.0094; HR_{1/4} 0.14; P_{1/4} 0.65). These data support the idea that there is a benefit to having some level of concordance between CD4⁺/CD8⁺ T-cell infiltration.

MHC-II expression, and MHC-II/MHC-I neoantigen ratios. To investigate differences in response dynamics between CD4⁺ and CD8⁺ T-cell mediated responses, the survival of responders MHC II- versus MHC I-reliant groups were compared.

Despite nonsignificant differences in response rates, MHC II-reliant responders had a significantly longer overall survival in both discovery and validation cohorts (FIGS. 8B and C, discovery P_{1/4} 0.0073; validation P_{1/4} 0.0398), consistent with reports that CD4⁺ T-cell-based immune responses are tumor autonomous and therefore more difficult to evade in the long term. FIG. 8B and FIG. 8C are Kaplan-Meier curves of overall survival of responders only in MHC II-reliant patients vs. MHC I-reliant patients for discovery and validation cohorts.

This observation was not solely reliant on one cohort or cancer type, as MHC reliance groupings were found to be balanced across all cohorts except Van Allen, and the findings were unchanged upon removal of the Van Allen cohort from this analysis.

Finally, it was wanted to know if differences in MHC reliance could translate to differences in pathways of immune evasion. ICs are commonly overexpressed to suppress an active immune response. Currently, of the many checkpoints identified in the tumor microenvironment only PDL1 positivity in tumor sections is approved as a biomarker of ICB response, albeit its predictive value is modest. To investigate whether differences might exist about which checkpoints correlate with a beneficial antitumor immune response under different MHC reliance conditions, the relationship between the expression of individual checkpoint genes and PFS post-ICB treatment by univariable Cox PH analysis was evaluated. Checkpoint genes with antibody inhibitors undergoing clinical trials (PDL1, CTLA4, LAG3, TIGIT, TIM3, IDO1, and OX40) were focused on. When split by MHC reliance grouping, higher LAG3 expression was associated with benefit from ICB in the MHC II-reliant group. To adjust for potentially confounding effects of the correlated expression of canonical immune checkpoint genes, a multivariable analysis was performed centered on LAG3, PDL1, and CTLA4. It was found that high PDL1 expression was generally associated with longer survival post-ICB treatment in MHC I-reliant patients (FIG. 8D, discovery P_{1/4} 0.026; validation P_{1/4} 0.062), CTLA4 expression with longer survival in balanced patients (FIG. 8D, discovery P_{1/4} 0.054; validation P_{1/4} 0.006), and LAG3 with longer survival in MHC II-reliant patients (FIG. 8D, discovery P_{1/4} 0.014; validation P_{1/4} 0.002). There was no association of checkpoint gene expression with the MHC reliance category, and some autocorrelation between LAG3, PDL1, and CTLA4 were observed. FIG. 8D illustrates HRs of key checkpoint molecule expression levels colored by MHC reliance status. Age, sex, and cohort-specific covariates are included in multivariate analysis. Error bars represent 95% CI.

Among MHC II-reliant patients, higher expression of LAG3 was associated with significantly longer overall survival in both discovery and validation cohorts (FIGS. 8E and 8F, discovery P_{1/4} 0.0018; validation P_{1/4} 0.0345). LAG3 is thought to play a prominent role in CD4⁺ T-cell regulation and may be a primary marker of activation Our results may, therefore, reflect a key role for LAG3 as a mediator of CD4⁺T-cell-based response to ICB therapy. FIGS. 8E and 8F illustrate Kaplan-Meier curves of LAG3 above/below median expression in MHC II-reliant patients in discovery and validation cohorts. *, P≤0.05; **, P≤0.01. Abbreviations include NR, nonresponders; ns, nonsignificant; OS, overall survival; and R, responders.

### Discussion

ICB has emerged as a potent anticancer therapy; however, the fraction of patients who benefit from treatment remains low. To improve the success of ICB, it is of the utmost importance to understand which factors govern the potential to respond via the immune system. Here, a machine learning framework was used to study somatic and germline biomarkers of response to ICB in human cohorts. Both feature types were extracted from paired tumor-normal WES data across eight ICB-treated human studies. Germline immune eQTL biomarkers, whereas relatively new, show promise to capture complementary information from somatic features, and XGBoost models trained to predict a cIC index using both feature types performed better at predicting ICB response across different tumor types. When patients with additional available RNAseq data were interrogated, it was found that the survival benefit of an immune hot microenvironment was contingent upon having a high cIC index score, that there was no response in patients with a low cIC index score, and that this was driven by germline features. This supports the notion that heritable differences in immune-cell function determine the effectiveness of an immune response once immune cells have reached the tumor. Furthermore, patients with a high cIC index score who failed to respond often had a “cold” TIME. This suggests that transcriptomic profiling might be useful as a supplemental prognostic tool of ICB response in high cIC index patients and that the cIC index score serves as a general proxy for clinical response to the immune invigorating effect of ICB.

To gain further insight about how various biomarkers relate to ICB response potential, techniques were used for interpreting machine learning models and studied important features and feature interactions that drove model predictions. The strongest interaction involved an interplay between an SNP associated with increased infiltration of T-follicular helper cells (T_{FH}QTL) and MHC-I damage. Specifically, a beneficial effect of the T_{FH}QTL on rates of response was observed, independent of the deleterious effect of MHC-I damage. T_{FH} cells are the specialized subset of CD4⁺ T cells that help B cells produce antibodies in germinal centers. T_{FH} cells are normally located in secondary lymphoid organs at a close distance from B cells. However, there is increasing evidence that T_{FH} cells are part of TLS, intra-tumor organized clusters of immune cells including B and T cells and dendritic cells mimicking germinal centers in secondary lymphoid organs TLS are an increasingly common finding in cancer, and are linked with better prognosis increased infiltration by T_{FH} cells and TLS formation are a source of helper factors beneficial to both CD8⁺ and CD4⁺ T cells. Indeed, the number of TLS distinguishes ICB responders from nonresponders.

MHC-I damage on cancer cells inherently hampers the cytotoxic function of CD8⁺ T cells, yielding low response rates. It was found that response rates were rescued when patients had both the T_{FH}QTL and MHC-I damage, suggesting that rescue mechanisms of ICB response may be shifted toward MHC-II mediated immunity (MHC-II reliance). Using individual-level information about the ratio of neoantigens with binding affinity for MHC-I and MHC-II, patients were allocated to either an MHC I- or MHC II-reliant group. That these groupings may initiate and sustain differential immune mechanisms in response to ICB is strengthened by the observation that MHC-II reliance promotes higher infiltration of CD4⁺ T cells and more durable clinical responses to ICB, potentially reflecting a direct effect on longterm memory CD4⁺ T-cell responses. In contrast, MHC I-reliant responses, which are centered on CD8⁺ T cells, are possibly more transient in the absence of CD4⁺ T-cell help.

The association of pretreatment checkpoint gene expression levels were examined with ICB response, which was predominantly anti-PD1/anti-PDL1 treatment in the cohorts studied, it was found that PDL1 expression was associated with better ICB response in MHC I-reliant patients but not in MHC II-reliant patients, whereas the reverse was true for LAG3. In patients in whom immune evasion is mediated by overexpression of PD1/PDL1, anti-PD1/anti-PDL1 therapies can be remarkably effective. In contrast, LAG3 has MHC-II as its major ligand, and it is widely regarded as a negative regulator of CD4⁺ T-cell activation. Higher expression of LAG3 could therefore indicate an effective ongoing MHC II-reliant antitumor response pre-ICB treatment. In the analysis, LAG3⁺ patients had better survival in the MHC II-reliant group, suggesting that MHC-II-driven immunity can support an effective response to antiPD1/anti-PDL1 and that this could potentially be further amplified by an anti-LAG3 therapy. However, the lack of association of PDL1 expression with response in the MHC II-reliant group seems to suggest a mechanism independent of alleviating PDL1-based repression of CD8⁺T cells. A similar phenomenon has been observed in microsatellite instability colorectal cancers with B2M loss that paradoxically remain among the best responders to anti-PD1/anti-PDL1 therapy. It is intriguing to think that anti-PD1/anti-PDL1 can be beneficial even if PDL1 is not highly expressed or the MHC-I antigen presentation machinery is not functional. Recent data show that LAG3 also associates with the T-cell receptor (TCR)-CD3 complex in both CD4⁺ and CD8⁺T cells in the absence of binding to MHC-II, causing the dissociation of the tyrosine kinase Lek from the CD4 or CD8 co-receptors and loss of co-receptor-TCR signaling during T-cell activation. Our finding that LAG3 facilitates the CD4⁺T-cell responses during ICB treatment could be explained by the fact that both LAG3 and ICB target the proximal signaling of the TCR, even though the reasons this creates an advantage in MHC II-reliant patients remains unclear. Perhaps this reflects the fact that the adult peripheral repertoire is richer in CD4⁺ than in CD8⁺T cells. This bias may also explain the observation that patients with cancer vaccinated with neoantigens have a propensity to generate CD4⁺ T-cell responses.

The other implication is that the utility of each of these checkpoint genes as biomarkers of ICB response may be highly context-dependent. PDL1 expression was not associated with ICB response in MHC II-reliant patients responding via a CD4⁺T-cell axis of adaptive immunity. This could explain in part why PDL1 positivity is a surprisingly poor general predictor of response rates. Future efforts to refine biomarkers of ICB response could attempt to leverage widely available germline information as well as understand the context of a patient's MHC reliance status.

This study provides further evidence that CD4⁺T-cell responses engaged by MHC-II antigen presentation are a critical component of superior immune responses and points to an alignment of checkpoint-based evasion with the immune cell types dominating the response.

### Example 2—Additional Genes Incorporated into the Model

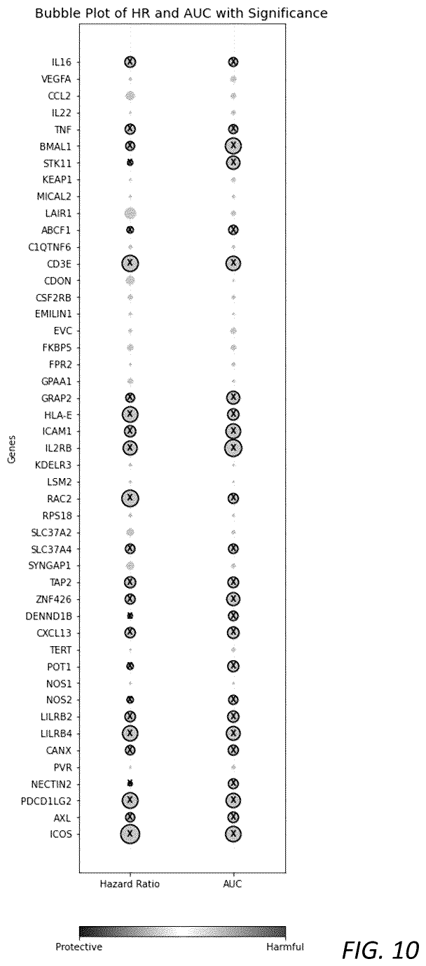

Additional genes were screened for associations between gene expression and response to immunotherapy. FIG. 10 illustrates a bubble plot of HR and area under the curve (AUC) of multiple genes. FIG. 10 shows an association between gene expression and response to immunotherapy if the expression value alone was used to predict response, both in CoxPH hazard ratio and AUC. As shown in FIG. 10, multiple genes had a significant association (marked by an x).

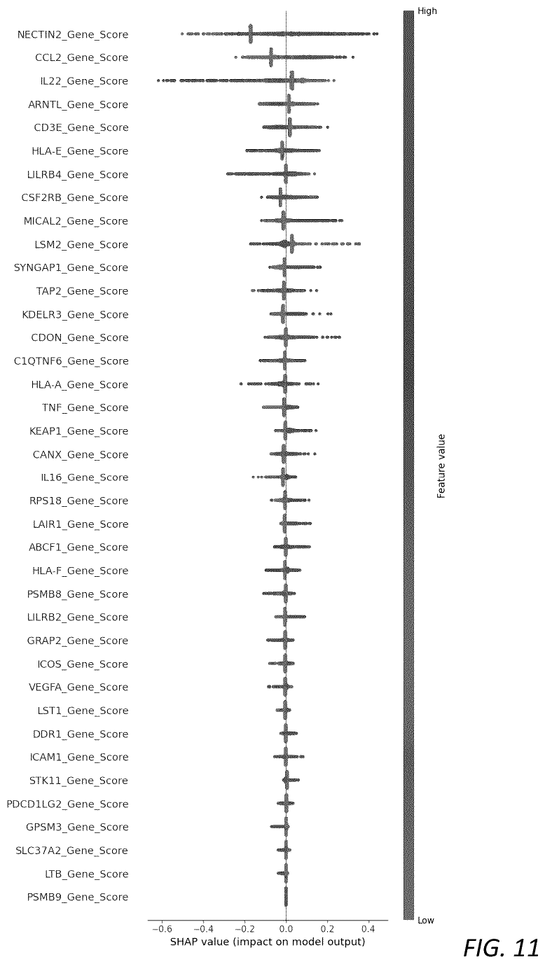

Feature importance for germline eQTLs for the additional genes in the model to predict immunotherapy response was investigated. FIG. 11 illustrates a plot of SHAP values versus feature values for the additionally screened genes. Approximately 1800 samples treated with ICI from a variety of different cancer types were used (mostly melanoma, lung cancer, urothelial/bladder cancer, kidney cancer, liver cancer, pancreatic cancer, head and neck cancer, breast cancer, gastric and colorectal cancer).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A computer implemented method for predicting a subject's response to an immunotherapy procedure to treat a cancer, the method comprising:
(a) receiving sequencing data of the subject;
(b) determining, using the sequencing data, a plurality of somatic features for the subject and a plurality of germline features for the subject; and
(c) generating, using the plurality of somatic features for the subject and the plurality of germline features for the subject, an immune checkpoint blockade (ICB) response score for the subject to represent a likelihood of response to an immunotherapy for the subject;
(d) comparing the ICB response score for the subject to an ICB response threshold value.

2. The method of claim 1, wherein determining a likelihood of response to an immunotherapy for the subject comprising comprises classifying the subject as an immunotherapy-responder based on a determination that the ICB response score is greater than the ICB response threshold.

3. The method of claim 1, wherein determining a likelihood of response to an immunotherapy for the subject comprising comprises classifying the subject as an immunotherapy-nonresponder based on a determination that the ICB response score is less than the ICB response threshold

4. The method of claim 1, wherein the sequencing data is whole-exome sequencing data.

5. The method of claim 1, wherein the plurality of sequencing feature values comprise a plurality of somatic feature values, a plurality of germline feature values, or both.

6. The method of claim 5, wherein the plurality of somatic feature values comprise at least one of the group consisting of: an immunoediting feature value, an immune escape feature value, an intratumoral heterogeneity feature value, a tumor mutational burden (TMB) feature value, a measure of immune evasion feature value, a damage of MHC-I alleles feature value, a DNA based T cell infiltration feature value, a somatic mutation of genes in an antigen presentation pathway feature value, an intratumoral heterogeneity feature value, and a fraction of TMB subclonal feature value.

7. The method of claim 5, wherein the plurality of germline features comprise at least one of the group consisting of: a single-nucleotide polymorphisms (SNP) associated with an immune infiltration levels feature value, a DNA repair and replication feature value, an immune signaling feature value, and an antigen processing and presentation feature value.

8. The method of claim 7, wherein the SNP associated with the immune infiltration levels is an SNP associated with FCGR2B, CTSS, FAM167A, FPR1, PDCD1, ITGB2, CTSW, FCGR3B, GPLD1, DCTN5, ERAP1, VAMP8, VAMP3, LYZ, ERAP2, DHFR, or TREX1 gene.

9. The method of claim 1, wherein the sequencing data comprises RNA sequencing data and the method comprises
(a) determining a tumor immune microenvironment (TIME) infiltration value from the RNA sequencing data to represent a composition of immune infiltrates.

10. The method of claim 9, wherein determining the immune checkpoint blockade (ICB) response score comprises use of at least one of the group consisting of at least one of the plurality of somatic features, at least one of plurality of the plurality of germline features, and the TIME infiltration value.

11. The method of claim 9, wherein the composition of immune infiltrates comprises at least one of the group consisting of: an effector CD8⁺ T cell infiltrate level, a joint B and CD4⁺ T cell level, and a target checkpoint expression.

12. The method of claim 1, wherein the cancer is selected from at least one of the group consisting of: a bladder cancer, a breast cancer, a cervical cancer, a colon cancer, a endometrial cancer, a esophageal cancer, a fallopian tube cancer, a gall bladder cancer, a gastrointestinal cancer, a head and neck cancer, a hematological cancer, a Hodgkin lymphoma, a laryngeal cancer, a liver cancer, a lung cancer, a lymphoma, a melanoma, a mesothelioma, a ovarian cancer, a primary peritoneal cancer, a salivary gland cancer, a sarcoma, a stomach cancer, a thyroid cancer, a pancreatic cancer, a renal cell carcinoma, a glioblastoma, and a prostate cancer.

13. The method of claim 12, wherein the cancer is a renal cell carcinoma (RCC), or a non-small cell lung cancer (NSCLC).

14. The method of claim 1, wherein the immunotherapy comprises administration of an immune checkpoint inhibitor.

15. The method of claim 14, wherein the immune checkpoint inhibitor is selected from at least one of the group consisting of: a PD-1 inhibitor, a PD-L1 inhibitor, and a CTLA-4 inhibitor.

16. The method of claim 1, comprising determining the sequencing features by:
(a) determining a feature importance for a multiplicity of sequencing features wherein the multiplicity of sequencing features comprise more features than the plurality of sequencing features,
(b) comparing the feature importance for each of the multiplicity of sequencing features to a feature importance threshold, and
(i) if the feature importance for one of the multiplicity of sequencing features meets the feature importance threshold, including the sequencing feature which meets the feature importance threshold in the plurality of sequencing features.

17. The method of claim 16, wherein determining the feature importance uses a Shapley Additive Explanations (SHAP) feature comparison model.

18. The method of claim 1, comprising determining, from the sequencing data, a number of mutations presented by a major histocompatibility complex class II (MHC-II) and a number of mutations presented by a major histocompatibility complex class I (MHC-I) of the subject, comparing the number of mutations presented by a major histocompatibility complex class II (MHC-II) and the number of mutations presented by a major histocompatibility complex class I (MHC-I) to an MHC mutation threshold, and, responsive to determining that the total number of mutations presented by the major histocompatibility complex class II (MHC-II) and the major histocompatibility complex class I (MHC-I) meets the MHC mutation threshold, determining a major histocompatibility complex (MHC) ratio of the subject.

19. The method of claim 18, wherein determining the major histocompatibility complex (MHC) ratio of the subject comprises, determining, from the sequencing data, a major histocompatibility complex (MHC) ratio of a total number of neoantigens presented by a major histocompatibility complex class II (MHC-II) of the subject divided by the total number of neoantigens presented by a major histocompatibility complex class I (MHC-I) of the subject, and, responsive to determining that the major histocompatibility complex (MHC) ratio of the subject meets a MHC ratio threshold, determining an immune checkpoint blockade (ICB) response score of the subject.

20. A computing system for determining whether a subject is at risk of having or developing a cancer, the system comprising:
a communication system configured to communicate over at least one data network with another computing device;
one or more processors; and
memory storing instructions that, when executed by the processors, cause the processors to perform operations comprising:
(a) receiving sequencing data of the subject;
(b) determining, using the sequencing data, a plurality of somatic features for the subject and a plurality of germline features for the subject; and
(c) generating, using the plurality of somatic features for the subject and the plurality of germline features for the subject, an immune checkpoint blockade (ICB) response score for the subject to represent a likelihood of response to an immunotherapy for the subject;
(d) comparing the ICB response score for the subject to an ICB response threshold value.

21. The method of claim 1, wherein determining a likelihood of response to an immunotherapy for the subject comprising comprises classifying the subject as an immunotherapy-responder based on a determination that the that the ICB response score is greater than the ICB response threshold.

22. The method of claim 1, wherein determining a likelihood of response to an immunotherapy for the subject comprising comprises classifying the subject as an immunotherapy-nonresponder based on a determination that the that the ICB response score is less than the ICB response threshold

23. A method for treating a subject that has been diagnosed with a cancer, the method comprising:
(a) receiving sequencing data of the subject;
(b) determining, using the sequencing data, a plurality of somatic features for the subject and a plurality of germline features for the subject; and
(c) generating, using the plurality of somatic features for the subject and the plurality of germline features for the subject, an immune checkpoint blockade (ICB) response score for the subject to represent subject to represent a likelihood of response to an immunotherapy for the subject;
(d) comparing the ICB response score for the subject to an ICB response threshold value, and
(e) administering to the subject the immunotherapy.
